SEQUENCE LISTING (17 sequences)

SEQ ID NO: 1 - protein, 12 aa, Hepatitis B virus
PLSSIFSRIGDP

SEQ ID NO: 2 - protein, 12 aa, Hepatitis B virus
PISSIFSRIGDP

SEQ ID NO: 3 - protein, 12 aa, Hepatitis B virus
PISSIFSRTGDP

SEQ ID NO: 4 - protein, 12 aa, Hepatitis B virus
HISSISARTGDP

SEQ ID NO: 5 - protein, 12 aa, Peking duck hepatitis virus
LLNQLAGRMIPK

SEQ ID NO: 6 - protein, 12 aa, Peking duck hepatitis virus
TIDHVLDHVQTM

SEQ ID NO: 7 - protein, 12 aa, heron hepatitis virus
TIQHVMDHIDSV

SEQ ID NO: 8 - protein, 12 aa, woodchuck hepatitis virus
TLSPVVPTVSTI

SEQ ID NO: 9 - protein, 12 aa, ground squirrel hepatitis virus
TLSPVVPTVSTT

SEQ ID NO: 10 - protein, 12 aa, Unknown
  polypeptide which acts as absorption-enhancing agent includes in a preferred embodiment a sequence covered by this general formula
  VARIANT (1): Xaa = any amino acid
  VARIANT (2): Xaa = hydrophobic amino acid
  VARIANT (3): Xaa = hydrophilic amino acid
  VARIANT (4): Xaa = hydrophilic amino acid
  VARIANT (5): Xaa = hydrophobic amino acid
  VARIANT (6): Xaa = any amino acid
  VARIANT (7): Xaa = any amino acid
  VARIANT (8): Xaa = hydrophilic amino acid
  VARIANT (9): Xaa = any amino acid
  VARIANT (10): Xaa = any amino acid
  VARIANT (11): Xaa = hydrophilic amino acid
  VARIANT (12): Xaa = any amino acid
XXXXXXXXXXXX

SEQ ID NO: 11 - protein, 12 aa, Unknown
  polypeptide which acts as absorption-enhancing agent includes in a preferred embodiment a sequence covered by this general formula
  VARIANT (1): Xaa = any amino acid
  VARIANT (2): Xaa = hydrophobic amino acid
  VARIANT (6): Xaa = any amino acid
  VARIANT (7): Xaa = any amino acid
  VARIANT (9): Xaa = any amino acid
XXSSIXXRXGDP

SEQ ID NO: 12 - protein, 12 aa, Artificial Sequence
  polypeptide which acts as absorption-enhancing agent includes in a preferred embodiment a sequence covered by this general formula
  VARIANT (3): Xaa = hydrophilic amino acid
  VARIANT (6): Xaa = any amino acid
  VARIANT (9): Xaa = any amino acid
  VARIANT (10): Xaa = any amino acid
  VARIANT (11): Xaa = hydrophilic amino acid
  VARIANT (12): Xaa = any amino acid
TIXHVXDHXXXX

SEQ ID NO: 13 - protein, 12 aa, Artificial Sequence
  polypeptide which acts as absorption-enhancing agent includes in a preferred embodiment a sequence covered by this general formula
  VARIANT (12): Xaa = any amino acid
TLSPVVPTVSTX

SEQ ID NO: 14 - DNA, 69 bp, Artificial Sequence
  PCR primer
gggaagctttcaagggtccccaatcctcgagaagattgacgataaggggtttcggaggtaacctgtaag

SEQ ID NO: 15 - DNA, 30 bp, Artificial Sequence
  PCR primer
gggaagctttcagtttcggaggtaacctgt

SEQ ID NO: 16 - DNA, 30 bp, Artificial Sequence
  PCR primer
gggggatccatgagctacaacttgcttgga

SEQ ID NO: 17 - DNA, 66 bp, Artificial Sequence
  PCR primer
gggggatcccccttatcgtcaatcttctcgaggattggggaccctatgagctacaacttgcttgga

(19) United States
(12) Patent Application Publication
(10) Pub. No.: US 2007/0172516 A1
(43) Pub. Date: Jul. 26, 2007

(54) Increasing of the resorption of substances via skin and mucous membranes

(75) Inventors: Trutz Podschun, München (DE); Peter Hans Hofschneider, München (DE); Eberhard Hildt, Berlin (DE)

Correspondence Address: SEED INTELLECTUAL PROPERTY LAW GROUP PLLC, 701 FIFTH AVE, SUITE 5400, SEATTLE, WA, 98104, US

(73) Assignee: ProCom Biotechnologische Produktions GmbH, Munchen (DE)

(21) Appl. No.: 10/526,846

(22) PCT Filed: Sep. 3, 2003
(86) PCT No.: PCT/EP03/09788

§ 371 Date: May 17, 2006

(30) Foreign Application Priority Data
Sep. 4, 2002 (DE) 102 40 894.7

(51) Int. Cl. A61K 39/12; A61K 38/21; A61F 13/02; A61K 38/10

(52) U.S. Cl. 424/448; 514/14; 424/85.4; 424/204.1; 977/802

(57) ABSTRACT

The invention relates to the increasing of the resorption of substances via skin and mucous membranes. The invention also relates to substances having an increased capability of being resorbed by skin and mucous membranes, and to pharmaceutical compositions containing substances of this type.

The invention relates to increasing the absorption of substances via the skin and mucous membranes. The invention further relates to substances having an enhanced ability to be absorbed by the skin and mucous membranes, and to pharmaceutical compositions comprising such substances.

The administration of biologically and therapeutically active substances by parenteral administration (e.g. intravenous, intramuscular and subcutaneous injection) is frequently regarded as the most suitable type of administration if the intention is to achieve a rapid and strong systemic effect, and the active substance is absorbed only slightly or not at all by the body or is inactivated in the gastrointestinal tract or through metabolism in the liver.

However, administration by injection has a number of disadvantages. Thus, the use of sterile syringes and needles or other mechanical devices is necessary, and pain, irritation and infections may occur, especially in the case of repeated injections. Moreover, injections should be administered only by trained people.

It is known that certain medicaments can be administered to a patient transdermally (percutaneously, via the—uninjured—skin) or transmucosally (via the mucous membranes). This administration essentially comprises the application of the medicament to the surface of the skin and/or of the mucosa and penetration by the medicament through the skin or mucosa into the patient's bloodstream.

Cutaneous or mucosal administration is interesting since it is possible in this way to generate a local and a systemic effect of a medicament. This type of administration can also be of interest as an alternative to parenteral administration if a rapid onset of an effect of the administered medicament is necessary.

Noninvasive administration moreover spares the physician and patient the inconveniences and risks associated with injections and infusions, and can also be performed by untrained people, i.e. even by the patient himself. This type of administration of medicaments is therefore associated with greater patient compliance than invasive techniques. This is true in particular of topical (local) or enteral administration, i.e. administration by the oral or rectal route.

Topical administration of systemically acting substances moreover has a significant advantage compared with cases in which oral absorption of the substance is poor, gastric intolerance occurs or the substance is metabolized in the liver immediately after absorption. A further advantage in these cases is that a systemic effect can be achieved by topical administration with a lower dose than that necessary for oral administration.

However, the skin and mucous membranes exert a physical and physiological barrier which must be overcome on administration of medicaments intended to reach internal tissues. Orally administered medicaments must moreover be resistant to the low pH and the digestive enzymes in the gastrointestinal tract.

Transdermal or transmucosal administration is therefore suitable only for medicaments which are absorbed well by the skin or mucosa.

The absorption rate and the fraction absorbed, i.e. the ratio of the absorbed portion to the administered amount, and ultimately the blood plasma levels which can be reached, i.e. the bioavailability of an active ingredient, depend besides other factors inter alia on sufficient solubility in water, other chemical properties of the substance and the physiological circumstances at the sites of administration and absorption. Many active pharmaceutical ingredients are extremely large and virtually impermeable for the skin and mucous membranes. In addition, absorption via mucous membranes is difficult for many active pharmaceutical ingredients because of their poor solubility in water or insolubility in water, thus conflicting with administration thereof via precisely these mucous membranes, for example by the enteral (oral and rectal), nasal, buccal, vaginal or urethral route.

Attempts have therefore been made to increase the percutaneous or transmucosal absorption of medicaments, i.e. a larger amount of the substance must penetrate through the skin or mucous membrane in a particular time. Substances which increase the absorption or the transport of molecules of low absorbability across biological membranes and thus increase the bio-availability of these molecules are known as absorption enhancers (Lee et al., Critical Reviews in Therapeutic Drug Carrier Systems 8, 91, 1991).

Absorption enhancers have been added to medicaments in order to increase the absorption thereof via the skin or mucous membranes. In this connection, these compounds increase the rate of permeation of the medicament through the skin or mucous membranes.

Examples of such absorption enhancers are alcohols and glycols (U.S. Pat. No. 5,296,222), urea derivatives, hyaluronic acids, N,N-dimethylformamide (DMF) and dimethyl sulfoxide (DMSO), terpenes (DE-A-10053383), bile acid salts (JP-A-59-130820), chelators (Cassidy and Tidball, J. Cell. Biol. 32, 685, 1967), surfactants (JP-A-4-247034, George et al., J. Infect. Dis. 136, 822, 1977), salts of fatty acids (U.S. Pat. Nos. 4,476,116 and 6,333,046), synthetic hydrophilic and hydrophobic compounds, biodegradable polymeric compounds and glycyrrhizic acid salts (JP-A-2-042027; U.S. Pat. No. 6,333,046).

Various mechanisms have been proposed for the action of absorption enhancers. These mechanisms of action include, at least for protein and peptide medicaments, (1) a reduction in the viscosity and/or elasticity of the mucous membranes, (2) a facilitated transcellular transport by increasing the fluidity of the bilayer of membranes and (3) an increase in the thermodynamic activity of medicaments (Lee et al., Critical Reviews in Therapeutic Drug Carrier Systems 8, 91, 1991).

However, at present, scarcely any absorption-enhancing product is available on the market. The reasons for this include the low efficacy and safety in relation to irritation and damage to mucous membranes, the unpleasant taste and odor, etc.

Problems arise for example in relation to the ratio between the enhancing action and the concentration of the absorption enhancer in the preparation. In the case of DMSO, the absorption-enhancing effect depends mainly on its concentration and it is thought that it is virtually ineffective at a concentration below 50%. In addition, it shows disadvantageous effects on the eyes and also displays side effects relating to the skin. The absorption-enhancing action of urea derivatives, hyaluronic acids, N,N-dimethylformamide and surfactants is low compared with dimethyl sulfoxide.

Nor do all absorption enhancers increase the absorption of all medicaments. The absorption enhancer must therefore be matched to the particular medicament.

Moreover, known absorption enhancers are frequently mucosal irritants or are unsuitable because of their unpleasant odor or taste, frequently lead to pain and lacrimation even after a single administration, or lead to irritation and inflammation of the mucosa after multiple uses. This applies for example to derivatives of fusidic acid, bile acids, surfactants and various glycols (polyethylene glycol, polypropylene glycol).

Moreover, many of these absorption enhancers lead to damage to the absorbing tissues and it has in fact been suggested that damage to the mucosa caused by these substances is the reason for an improved absorption (LeCluyse and Sutton, Advanced Drug Delivery Reviews 23, 163, 1997).

The known enhancers of transdermal or transmucosal absorption are therefore inadequate in terms of their action and safety.

Also known in the prior art are the so-called transferosomes (DE 41 07 152, DE 41 07 153 and DE 44 47 287). They are used for noninvasive administration of suitable active ingredients through the skin. Transferosomes are distinguished from other liposomes described for topical use through an improved penetration ability. Transferosomes are usually much larger than conventional micellar carrier formulations and are therefore subject to different diffusion laws. The increased penetration ability is achieved through their specific composition which makes them sufficiently elastic (hyperflexible) to be able to overcome the constrictions in the barrier, e.g. in the skin.

The object of the invention is to improve the absorbability of substances difficult to absorb through the skin and mucous membranes, in order thus to improve the fraction of these substances absorbed.

The intention is thereby to enable the noninvasive use of substances which are normally absorbed only poorly, or not at all, by skin or mucous membranes without at the same time requiring great technical elaboration and large consumption of active ingredients.

This object is achieved according to the invention by the subject matter of the claims.

The object of the invention is achieved by coupling an agent which increases the absorption of a substance through the skin or mucosa to the substance and thus increasing the bioavailability of the substance.

The combination according to the invention of a substance and an agent enhancing absorption surprisingly makes it possible to improve the fraction absorbed and/or permeation of substances via the skin and mucous membranes, which have to date been regarded as poorly absorbable or nonabsorbable.

The increasing action (enhancing action) of agents on the absorption of substances via or through the skin or mucous membranes makes it possible to obtain forms for administration of therapeutic, diagnostic or cosmetic substances via the skin and mucosa such as the nasal mucosa, eye mucosa, tracheal/bronchial/lung mucosa, rectal mucosa, the mucosa of the genital tract, the oral mucosa, the gastrointestinal mucous membranes, the vaginal mucosa or else the ureteral mucosa even for substances which have to date been poorly absorbable or nonabsorbable.

The agent increasing the absorption acts in this case as absorption enhancer to increase the bioavailability of the substance. Despite the poor original absorption and, associated therewith, low bioavailability, it is thus possible to achieve a satisfactory absorption with all the therapeutic consequences, and the dosage of the substance can where appropriate also be reduced by comparison with the conventional dosage, or an improved effect can be achieved if the dosage remains the same.

The invention thus relates in one aspect to a method for producing a percutaneous or transmucosal product comprising the coupling of a substance to at least one agent which increases the absorption of the substance through skin or mucosa.

The invention relates in a further aspect to a method for increasing the bioavailability of a substance on application to the skin or mucosa, comprising the coupling of the substance to at least one agent which increases the absorption of the substance through skin or mucosa.

The invention also relates to a method for increasing the ability of a substance to be absorbed by skin or mucosa on application thereto, comprising the coupling of the substance to at least one agent which increases the absorption of the substance through skin or mucosa.

The invention further relates to a method for increasing the permeation ability (penetration ability) of a substance for skin or mucosa, comprising the coupling of the substance to at least one agent which increases the absorption of the substance through skin or mucosa.

The invention relates in a further aspect to the substances obtainable by the methods of the invention and having increased bioavailability, increased ability to be absorbed by skin or mucosa, and/or increased permeation ability (penetration ability) and pharmaceutical compositions comprising one or more of these substances.

The invention further relates to the use of the substances obtainable by the methods of the invention and having increased bioavailability, increased ability to be absorbed by skin or mucosa, and/or increased permeation ability (penetration ability) and pharmaceutical compositions thereof for application to the skin or mucosa and for the treatment (including prophylaxis and cosmetic treatment) and/or diagnosis of disorders which are usually treated, prevented or diagnosed with these substances without the modification of the invention.

The invention further relates to methods for the treatment (including prophylaxis and cosmetic treatment) and/or diagnosis of a disorder in a patient, comprising the administration of a pharmaceutical composition which comprises the substances obtainable by the methods of the invention and having increased bioavailability, increased ability to be absorbed by skin or mucosa, and/or increased permeation ability (penetration ability), to the patient so that the concentration (local or systemic, preferably systemic) of the substance with increased bioavailability, increased ability to be absorbed by skin or mucosa, and/or increased permeation ability (penetration ability), is sufficient to treat, to prevent and/or to diagnose the disorder.

The invention relates in a further aspect to a method for elucidating a mucosal, dermatological and/or systemic effect of a substance, in particular an active pharmaceutical ingredient, which includes an administration via the skin or mucous membranes of a pharmaceutical composition which comprises the substances obtainable by the methods of the invention and having increased bioavailability, increased ability to be absorbed by skin or mucosa, and/or increased permeation ability (penetration ability), in a mucosally, dermally and/or systemically effective amount to a patient.

The absorption-enhancing agent may according to the invention be linked (coupled) covalently or noncovalently with a substance. A linkage is preferably a covalent linkage.

In one embodiment, a linker is present between the substance and the absorption-enhancing agent. The linker can preferably be cleaved, for example enzymatically or chemically, in particular by in vivo processes, so that the substance can be separated from the absorption-enhancing agent. The linker comprises in one embodiment a cleavable ester or carbamate functionality or a peptide which can be recognized by a proteinase such as a proteinase occurring in serum. In a particularly preferred embodiment, the substance is separated from the absorption-enhancing agent after absorption through the skin or mucosa.

In one embodiment, the absorption-enhancing agent is coupled more than once to the substance, i.e. at least 2, preferably 2 to 10, more preferably 2 to 5, even more preferably 2 to 3, in particular 2, absorption-enhancing agents, which may be identical or different, are coupled (covalently and/or noncovalently) to the substance. These multiply coupled absorption-enhancing agents may be linked separate from one another or in series with one another, where appropriate separated by a linker, as tandem constructs to the substance. This preferably achieves a greater bioavailability, ability to be absorbed by skin or mucosa, and/or increased permeation ability (penetration ability), than with a simple coupling of the absorption-enhancing agent.

In a preferred embodiment, the absorption-enhancing agent is a polypeptide or protein. The polypeptide or protein preferably includes a sequence derived from a virus and, in particular, a sequence derived from a surface protein of a virus, or a derivative or a part thereof. The term “virus” includes DNA viruses and RNA viruses, especially adenoviruses, adeno-associated viruses, vaccinia viruses, baculoviruses, hepatitis C viruses, hepatitis A viruses, influenza viruses, herpes viruses and hepadna viruses. Examples of the latter are HBV, WHV (“woodchuck hepatitis virus”), GSHV (“ground squirrel hepatitis virus”), RBSHV (“red-bellied squirrel hepatitis virus”), DHV (“Pekin duck hepatitis virus”) and HHV (“heron hepatitis virus”). In a particularly preferred embodiment, the peptide or protein includes a sequence derived from a hepatitis virus, hepadna virus or HIV, especially a hepatitis B virus, or a derivative or a part thereof. The peptide or protein preferably includes a sequence which is derived from antennapedia, which is derived from HIV tat or which is derived from VP22 of a herpes virus.

In a preferred embodiment, the term “virus” includes viruses which occur in humans, non-human primates or other animals, especially mammals (such as cow, horse, pig, sheep, goat, dog and cat), birds (such as, for example, chicken) or rodents (such as mouse and rat).

The polypeptide or protein which acts as absorption-enhancing agent includes in a preferred embodiment a sequence which is covered by the general formula below:
X1-X2-X3-X4-X5-X6-X7-X8-X9-X10-X11-X12,
in which X1, X6, X7, X9, X10 and X12 are variable, X2 and X5 are hydrophobic amino acid residues and X3, X4, X8 and X11 are hydrophilic amino acid residues. X7 is preferably a hydrophilic amino acid residue.

In particular embodiments, the polypeptide or protein which acts as absorption-enhancing agent includes this sequence with one or two amino acid residues, in particular one amino acid residue from X1 to X12, differing from this hydropathic profile.

Amino acid side chains with charged groups, hydrogen bond-forming groups or dipoles can be classified as hydrophilic. In contrast thereto, neutral organic amino acid side chains with a hydrocarbon character having no significant dipoles and not having the ability to form hydrogen bonds can be classified as hydrophobic.

The table below shows the hydropathic index of amino acid side chains according to Kyte and Doolittle, J. Mol. Biol. 157, 105, 1982:

| Amino acid | Hydropathic index |
|---|---|
| Isoleucine (Ile, I) | 4.5 |
| Valine (Val, V) | 4.2 |
| Leucine (Leu, L) | 3.8 |
| Phenylalanine (Phe, F) | 2.8 |
| Cysteine (Cys, C) | 2.5 |
| Methionine (Met, M) | 1.9 |
| Alanine (Ala, A) | 1.8 |
| Glycine (Gly, G) | −0.4 |
| Threonine (Thr, T) | −0.7 |
| Tryptophan (Trp, W) | −0.9 |
| Serine (Ser, S) | −0.8 |
| Tyrosine (Tyr, Y) | −1.3 |
| Proline (Pro, P) | −1.6 |
| Histidine (His, H) | −3.2 |
| Glutamic acid (Glu, E) | −3.5 |
| Glutamine (Gln, Q) | −3.5 |
| Aspartic acid (Asp, D) | −3.5 |
| Asparagine (Asn, N) | −3.5 |
| Lysine (Lys, K) | −3.9 |
| Arginine (Arg, R) | −4.5 |

Hydrophobic amino acids include according to the invention alanine, valine, leucine, isoleucine, tryptophan, phenylalanine and methionine. The hydrophilic amino acids include according to the invention glycine, serine, tyrosine, threonine, cysteine, aspartic acid, asparagine, glutamic acid, glutamine, lysine, arginine, histidine and proline. A variable amino acid residue may be any of the amino acids listed above.

X1 is preferably proline, histidine, leucine or threonine, more preferably proline or threonine, in particular proline. X2 is preferably alanine, valine, leucine or isoleucine, more preferably leucine or isoleucine, in particular leucine. X3 is preferably serine, asparagine, aspartic acid or glutamine, in particular serine. X4 is preferably serine, glutamine, histidine or proline, more preferably serine, histidine or proline, in particular serine. X5 is preferably alanine, valine, leucine or isoleucine, more preferably isoleucine or valine, in particular isoleucine. X6 is preferably phenylalanine, serine, alanine, leucine, methionine or valine, more preferably phenylalanine or valine, in particular phenylalanine. X7 is preferably serine, alanine, glycine, aspartic acid or proline, more preferably serine, aspartic acid or proline, in particular serine. X8 is preferably arginine, histidine or threonine, more preferably arginine or histidine, in particular arginine. X9 is preferably isoleucine, threonine, methionine or valine, more preferably isoleucine or valine, in particular isoleucine. X10 is preferably glycine, isoleucine, glutamine, aspartic acid or serine, more preferably glycine or serine, in particular glycine. X11 is preferably aspartic acid, proline, threonine or serine, more preferably aspartic acid or threonine, in particular aspartic acid. X12 is preferably proline, lysine, methionine, valine, isoleucine or threonine, in particular proline.

In a preferred embodiment, the polypeptide or protein which acts as absorption-enhancing agent includes an amino acid sequence which is covered by the general formula below:
(I) X1-X2-S-S-I-X6-X7-R-X9-G-D-P,
in which

X1 is a variable amino acid, preferably proline, histidine, leucine or threonine, more preferably proline or histidine, in particular proline,

X2 is a hydrophobic amino acid, preferably alanine, valine, leucine or isoleucine, more preferably leucine or isoleucine, in particular leucine,

X6 is a variable amino acid, preferably phenylalanine, serine, alanine, leucine, methionine or valine, more preferably phenylalanine or serine, in particular phenylalanine,

X7 is a variable amino acid, preferably serine, alanine, glycine, aspartic acid or proline, more preferably serine or alanine, in particular serine, and

X9 is a variable amino acid, preferably isoleucine, threonine, methionine or valine, more preferably isoleucine or threonine, in particular isoleucine.

In a further preferred embodiment, the polypeptide or protein which acts as absorption-enhancing agent includes an amino acid sequence which is covered by the general formula below:
(II) T-I-X3-H-V-X6-D-H-X9-X10-X11-X12,
in which

X3 is a hydrophilic amino acid, preferably serine, asparagine, aspartic acid or glutamine, in particular aspartic acid or glutamine,

X6 is a variable amino acid, preferably phenylalanine, serine, alanine, leucine, methionine or valine, in particular leucine or methionine,

X9 is a variable amino acid, preferably isoleucine, threonine, methionine or valine, in particular valine or isoleucine,

X10 is a variable amino acid, preferably glycine, isoleucine, glutamine, aspartic acid or serine, in particular aspartic acid or glutamine,

X11 is a hydrophilic amino acid, preferably aspartic acid, proline, threonine or serine, in particular serine or threonine, and

X12 is a variable amino acid, preferably proline, lysine, methionine, valine, isoleucine or threonine, in particular valine or methionine.

In a further preferred embodiment, the polypeptide or protein which acts as absorption-enhancing agent includes an amino acid sequence which is covered by the general formula below:
(III) T-L-S-P-V-V-P-T-V-S-T-X12,
in which

X12 is a variable amino acid, preferably proline, lysine, methionine, valine, isoleucine or threonine, in particular isoleucine or threonine.

In a further embodiment, the polypeptide or protein which acts as absorption-enhancing agent includes one of the amino acid sequences listed below, or an amino acid sequence derived therefrom:

(1) P-L-S-S-I-F-S-R-I-G-D-P;

(2) P-I-S-S-I-F-S-R-I-G-D-P;

(3) P-I-S-S-I-F-S-R-T-G-D-P;

(4) H-I-S-S-I-S-A-R-T-G-D-P;

(5) L-L-N-Q-L-A-G-R-M-I-P-K;

(6) T-I-D-H-V-L-D-H-V-Q-T-M;

(7) T-I-Q-H-V-M-D-H-I-D-S-V;

(8) T-L-S-P-V-V-P-T-V-S-T-I;

(9) T-L-S-P-V-V-P-T-V-S-T-T.

In the most preferred embodiment, the polypeptide or protein which acts as absorption-enhancing agent includes the amino acid sequence:

(1) P-L-S-S-I-F-S-R-I-G-D-P

The polypeptides or proteins which act as absorption-enhancing agents and which are described in the invention may also be derivatives thereof, in particular amino acid insertion variants, amino acid deletion variants and/or amino acid substitution variants. Amino acids are preferably replaced by others having similar properties such as hydrophobicity, hydrophilicity, electronegativity, volume of the side chain and the like (conservative substitution). Conservative substitutions relate in this connection for example to replacement of one amino acid by another, with both amino acids being listed in the same group below:
1. small aliphatic, nonpolar or slightly polar residues: Ala, Ser, Thr (Pro, Gly)
2. negatively charged residues and their amides: Asn, Asp, Glu, Gln
3. positively charged residues: His, Arg, Lys
4. large aliphatic, nonpolar residues: Met, Leu, Ile, Val (Cys)
5. large aromatic residues: Phe, Tyr, Trp.

Three residues are placed in parentheses because of their particular importance for the protein architecture. Gly is the only residue without a side chain and thus confers flexibility on the chain. Pro has an unusual geometry which greatly restricts the chain. Cys can form a disulfide bridge.

In one embodiment, from 1 to 6, preferably 1 to 4, more preferably 1 to 3, in particular 1 to 2, amino acids may be replaced in the polypeptides or proteins which act as absorption-enhancing agents and are described in the invention.

The polypeptides or proteins which act as absorption-enhancing agents and are described in the invention may also include non-naturally occurring amino acids such as D-amino acids, non-classical amino acids or chemical amino acid analogues. Non-classical amino acids and chemical amino acid analogues include in a non-restrictive manner α-aminobutyric acid, aminobutyric acids, aminohexanoic acids, aminopropionic acids, β-alanine, γ-carboxyglutamic acid, ornithine, norleucine, norvaline, hydroxyproline, sarcosine, citrulline, cysteic acid, t-butylglycine, t-butyl-guanine, phenylglycine, cyclohexylalanine, P-alanine, fluoroamino acids, ring-methylated phenylalanine, and the like. Each amino acid residue may be replaced by a non-classical amino acid or a chemical amino acid analogue. It is preferably possible in this way to increase the solubility, stability or absorption through the skin or mucosa.

The polypeptide or protein which acts as absorption-enhancing agent includes in a further embodiment an amino acid sequence or sequence derived therefrom which have a hydropathic profile which corresponds to one or more of the amino acid sequences listed below:

(1) P-L-S-S-I-F-S-R-I-G-D-P;

(2) P-I-S-S-I-F-S-R-I-G-D-P;

(3) P-I-S-S-I-F-S-R-T-G-D-P;

(4) H-I-S-S-I-S-A-R-T-G-D-P;

(5) L-L-N-Q-L-A-G-R-M-I-P-K;

(6) T-I-D-H-V-L-D-H-V-Q-T-M;

(7) T-I-Q-H-V-M-D-H-I-D-S-V;

(8) T-L-S-P-V-V-P-T-V-S-T-I;

(9) T-L-S-P-V-V-P-T-V-S-T-T.

The term “hydropathic profile which corresponds to an amino acid sequence” means according to the invention that amino acid residues which are in each case to be assigned to hydrophilic, hydrophobic or variable amino acid residues are present at corresponding positions of two or more amino acid sequences.

In a preferred embodiment, the polypeptide or protein which acts as absorption-enhancing agent includes an amino acid sequence which corresponds in at least 10, more preferably at least 11, in particular 12, amino acid residues to the hydropathic profile of these amino acid sequences (1) to (9), either singly or looking at two or more amino acid sequences together.

If the substance with which the peptide or protein which acts as absorption-enhancing agent is to be coupled is likewise a peptide or protein, the absorption-enhancing polypeptide or protein may be present at the N, C terminus, on a side chain and/or in the interior as insertion (internally) of the substance to be coupled. Peptides or proteins which comprise the absorption-enhancing agent at the N and/or C terminus can be prepared recombinantly by fusing a nucleic acid coding for the absorption-enhancing polypeptide or protein to the nucleic acid which codes for the peptide or protein to be coupled, and expressing the fused sequence, e.g. in a cell. A further possibility if the substance which is a peptide or protein is to comprise the absorption-enhancing agent internally is to insert the nucleic acid coding for the absorption-enhancing agent into the nucleic acid coding for the substance.

The invention also relates to such peptide/protein constructs and nucleic acids coding therefor and derivatives thereof.

These peptide/protein constructs and nucleic acids or derivatives thereof are preferably recombinant constructs and not peptides/proteins or nucleic acids which naturally comprise the polypeptides or proteins which act as absorption-enhancing agents and are described in the invention, or nucleic acids coding therefor, where the term “naturally” relates to a peptide, protein or a nucleic acid which is to be found in nature, e.g. in an animal or plant, without human intervention.

A coupling of a peptide/protein substance with a polypeptide or protein which acts as absorption-enhancing agent via the side chain(s) of the peptide/protein substance may take place for example via acidic amino acids and the amides thereof, such as aspartic acid, asparagine, glutamic acid and glutamine, or basic amino acids such as lysine and arginine, directly or via a linker.

It is possible according to the invention for any substance, inorganic or organic in nature, to be coupled to an agent which increases the absorption of the substance through skin or mucosa. The substance may as such be absorbed, poorly absorbed or not absorbed. The substance is preferably an active pharmaceutical ingredient whose transdermal or transmucosal absorption can be improved. The active pharmaceutical ingredient may be of animal or vegetable origin, and is preferably a pure substance of animal or vegetable origin, or may be of synthetic origin.

In a further embodiment, at least 2, preferably 2 to 4, more preferably 2 to 3, in particular 2, substances, which may be identical or different, are coupled together, and this conjugate is preferably coupled to at least 1, preferably 1 to 5, more preferably 1 to 3, even more preferably 1 or 2, in particular 1, identical or different absorption-enhancing agents. In a preferred embodiment, the substances and/or the absorption-enhancing agent(s) are coupled via linkers.

It is possible in this embodiment for therapeutic, prophylactic and/or diagnostic effects which derive from different substances to be achieved through administration of only one compound.

In a preferred embodiment, the substance which is coupled to the absorption-enhancing agent may have its native (i.e. naturally occurring and active) structure or a modified structure. The term “modified structure” means according to the invention any non-native structure of the substance. A modified structure includes for example a modified polypeptide or protein in which one or more modifications, in particular post-translational modifications, are absent and/or additionally present compared with the native polypeptide or protein. Modifications, in particular post-translational modifications, include in a non-limiting fashion glycosylations, oxidations of cysteine side chains, isomerizations of disulfide bridges and peptidyl-prolyl linkages, hydroxylations, carboxylations, acylations and the like.

In a further embodiment, the substance which is coupled to the absorption-enhancing agent may, before or after transdermal or transmucosal absorption, have an activity which corresponds to that of the native substance or is lower or higher. In various embodiments, the activity of the substance before or after transdermal or transmucosal absorption is less than 100%, less than 80%, less than 60% or less than 50%, of the activity of the native substance. In one embodiment, the substance has no activity, i.e. it is inactive compared with the native substance. In this embodiment, the substance can be employed in particular for immunization.

An active pharmaceutical ingredient may include according to the invention any biologically active substance which is selected from the group: analgesics, amino acids, anorectic agents, antibiotics, antiallergics, antiarrhythmics, anticholinergics, antidepressants, antidiabetics, antidotes, antiemetics, antiepileptics, antiinfectious agents, antigens, antihistamines and histamines, antihypertensives, anticoagulants, anticonvulsants, antibodies, anti-mycotics, antineoplastics, antiinflammatory agents, antipsorics, antipyretics, antiseptics, antitumor agents, antitussives (asthma remedies) and other agents related to breathing, antiviral and anticancer agents, antiworm agents, anxiolytics, ophthalmic medicaments (including antiglaucoma agents), beta-blockers, imaging agents, blood factors, bronchodilators, chaperones, chemokines, chemotherapeutics, cholesterol-lowering agents, cytokines, dermatological agents, diagnostic agents, diuretics and antidiuretics, DNA-modifying agents, enzymes, dietary supplements, fibrinolytics, gaba antagonists, gastrointestinal hormones and derivatives thereof, sex hormones, glutamate antagonists, glycine antagonists, hematopoietics, hormones, hypnotics, pituitary hormones and derivatives thereof, hypothalamus hormones and derivatives thereof, inhibitors of a signal transduction pathway, integrins, interferons, interleukins, inverse peptides, cardiotonics, kinase inhibitors, contrast agents, contraceptives, corticosteroids and derivatives thereof, cosmetics, leucotrienes, local anesthetics, lymphokines, MHC/HLA molecules, antianginal agents, antidementia or antiparkinson agents, antihyper-lipidemia agents, antihypoglycemia agents, antimigraine agents, monokines, muscle relaxants, Mx proteins, anesthetics, adrenal hormones, pancreatic hormones and derivatives thereof, parasympathomimetics, para-sympatholytics, peptidomimetics, plasmids, potency-increasing agents, promoters, prostaglandins, psychoactive drugs, recombinant proteins, repressors, thyroid hormones and derivatives thereof, sedatives, spasmolytics, steroid hormones, sympathomimetics, terminators, therapeutic agents for osteoporosis, tranquilizers, thrombolytics, vaccines, vasoconstrictors, vasodilators, vitamins, cell adhesion molecules and the like.

Analgesics include in a nonrestrictive manner fentanyl, morphine, tramadol, hydrocodeine, methadone, lidocaine, diclofenac, paverine and the like.

Antiarrhythmics include substances which influence the cardiac excitation process in order preferably to treat cardiac arrhythmias. One example of a class of antiarrhythmics are the beta-blockers such as, for example, propanolol, alprenolol, timolol, nadoxolol and the like.

Antibiotics, antiinfectious agents, antimycotics and antiviral agents include in a nonrestrictive manner tetracyclines, tetracycline-like antibiotics, erythromycin, 2-thiopyridin N-oxide, halogen compounds (preferably iodine-containing compounds such as iodine-polyvinylpyrrolidone complex), β-lactam compounds such as penicillin compounds (e.g. penicillin G or V), cephalosporins, sulfonamide compounds, aminoglycoside compounds (such as streptomycin), amphothericin B, 5-iodo-2-deoxyuridine, gramicidin, nystatin and the like.

Antidiuretics and diuretics include in a nonrestrictive manner desmopressin, vasopressin, furosemide and the like.

Nonlimiting examples of antiemetics include pipamazine, chlorpromazine, dimenhydrinate, meclozine, metoclo-pramide and the like.

Antihistamines include compounds which inhibit the effects of histamine. Nonlimiting examples thereof are 3-(2-aminoethyl)pyrazole, cimetidine, cyproheptadine hydrochloride and the like.

Antihypertensives, antianginal agents and vasodilators include in a nonrestrictive manner compounds such as clonidine, α-methyldopa, nitroglycerine, polynitrates of polyalcohols (e.g. erythritol tetranitrate and mannitol hexanitrate), papaverine, dipyridamole, nifedipine, diltiazem and the like.

Antiinflammatory agents include in a nonrestrictive manner steroidal and non-steroidal antiinflammatory drugs. Examples thereof are cortisone, hydrocortisone, betamethasone, dexamethasone, prednisolone, ibuprofen, aspirin, salicylic acid, flumethasone, fluprednisolone, aminopyrine, antipyrine, fluprofen and derivatives thereof.

Antitussives include in a nonrestrictive manner compounds such as cromoglycate and derivatives thereof, beclomethasone, budesonide, salbutamol, mometasone, terbutaline and the like.

Contraceptives relates to compounds which in female patients prevent ovulation or implantation of the fertilized egg in the placenta or in male patients prevent sperm maturation. Nonlimiting examples thereof are ethinylestradiol, medroxyprogesterone acetate and antiprogestins (such as, for example, RU 486).

Antimigraine agents include in a nonrestrictive manner heparin, hirudin and the like.

Examples of muscle relaxants include in a nonrestrictive manner cyclobenzapyrine hydrochloride, diazepam, alcuronium, vecuronium, succinyldicholine and the like.

Anesthetics and local anesthetics include in a nonrestrictive manner benzocaine, procaine, propoxycaine, dibucaine, lidocaine, naloxone, naltrexone and derivatives thereof.

Peptidomimetics and inverse peptides include peptide-like compounds which act as peptides but do not have the typical peptide structure. A nonlimiting example thereof is a peptide analogue which, in contrast to its native peptide, is composed only of D-amino acids.

Potency-increasing agents include in a nonrestrictive manner those active pharmaceutical ingredients which increase the libido of a patient and/or lead to a prolonged sexual performance. Examples of potency-increasing agents are those which increase NO synthesis in the patient (e.g. sildenafin).

Steroid hormones are hormones derived from cholesterol. Steroid hormones include in a nonrestrictive manner gestagens (such as progesterone), corticoids which include glucocorticoids (such as cortisone and cortisol) and mineralocorticoids (such as aldosterone), sex hormones such as androgens (e.g. testosterone) and estrogens (e.g. estrone and estradiol) and derivatives thereof (e.g. dexamethasone, betamethasone, prednisolone, beclomethasone, mometasone and the like).

The active ingredient may also be a nucleic acid or “antisense” nucleic acid or a derivative thereof.

“Antisense” molecules or “antisense” nucleic acids can be used for regulating, in particular reducing, the expression of a nucleic acid. The term “antisense molecule” or “antisense nucleic acid” relates according to the invention to an oligonucleotide which is an oligoribonucleotide, oligodeoxyribonucleotide, modified oligoribonucleotide or modified oligodeoxyribonucleotide and which, under physiological conditions, hybridizes onto DNA which includes a particular gene, or mRNA of this gene, thus inhibiting the transcription of this gene and/or the translation of this mRNA. An “antisense molecule” also includes according to the invention a construct which comprises a nucleic acid or part thereof in reverse orientation in relation to its natural promoter. An antisense transcript of a nucleic acid or of a part thereof may enter a duplex molecule with the naturally occurring mRNA which specifies the enzyme, and thus prevent accumulation of or translation of the mRNA into the active enzyme.

In preferred embodiments, an oligonucleotide is a “modified” oligonucleotide. In these cases, the oligonucleotide may be modified, in order for example to increase its stability or therapeutic efficacy, in a wide variety of ways without impairing its ability to bind to its target. The term “modified oligonucleotide” means according to the invention an oligonucleotide in which (i) at least two of its nucleotides are linked together by a synthetic internucleoside linkage (i.e. an internucleoside linkage which is not a phosphodiester linkage) and/or (ii) a chemical group which does not normally occur in nucleic acids is covalently linked to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also includes oligonucleotides having a covalently modified base and/or sugar and oligonucleotides which comprise non-naturally occurring nucleotides and/or nucleotide analogues. “Modified oligonucleotides” include for example oligonucleotides having sugar residues which are covalently linked to organic groups which have a low molecular weight and which are not a hydroxyl group in the 3′ position and not a phosphate group in the 5′ position. Modified oligonucleotides may include for example a 2′-O-alkylated ribose residue or another sugar in place of ribose such as arabinose. Modified oligonucleotides may also comprise modified bases and/or base analogues such as, for example, 7-deazaadenosine, 7-deazaguanosine, isoguanosine, 2-thiopyrimidine, isocytidine, universal base and the like.

The active ingredient may also be a gene, a gene-correcting oligonucleotide, an aptameric oligonucleotide, triple helix nucleotide or a ribozyme.

The active ingredient may also be a polypeptide or protein or a derivative thereof. It may moreover be a conjugate of a plurality of peptides or proteins which have been coupled together chemically or genetically. The peptides or proteins used according to the invention may be derived from a natural source or be recombinantly or chemically synthesized substances. The polypeptides and proteins employed according to the invention are preferably isolated. The terms “isolated protein” or “isolated polypeptide” mean that the protein or polypeptide is separated from its natural environment. An isolated protein or polypeptide may be in an essentially purified state. The term “essentially purified” means that the protein or polypeptide is essentially free of other substances with which it is present in nature or in vivo.

The polypeptides or proteins which can be employed according to the invention include in a nonlimiting manner antibiotics, hematopoietics, antiinfectious agents, antidementia agents, antiviral agents, antitumor agents, antipyretics, analgesics, antiinflammatory agents, antiallergics, anti-depressants, antipsorics, psychoactive drugs, cardiotonics, antiarrhythmics, vasodilators, antihypertensives, antidiabetics, anticoagulants, cholesterol-lowering agents, therapeutic agents for osteoporosis, hormones, vaccines and the like, and the polypeptides and proteins which have been described above as active pharmaceutical ingredients.

Particularly preferred peptides or proteins include cytokines, peptide hormones, growth factors, factors of the cardiovascular system, factors of the central and peripheral nervous system, factors of the gastrointestinal system, factors of the immune system, enzymes and vaccines.

Lymphokines, monokines, hematopoietic factors and the like are particularly preferred.

Lymphokines include interferons (e.g. α-, β- and γ-interferon and their subtypes, including IFN-α-2a, IFN-α-2b and IFN-α-n3), interleukins (e.g. interleukin 1-17) and the like.

“Interferon” is a term which generally includes a group of glycoproteins and proteins from vertebrates which are known to have various biological activities such as antiviral, antiproliferative and immunomodulating activities. The term “interferon” relates according to the invention to native and recombinant proteins, and to proteins which are expressed in the eukaryotic cells, especially mammalian cells, as well as prokaryotic cells. The term “interferon” thus includes in relation to IFN-β both IFN-β-1a and IFN-β-1b.

Interferons are secretory proteins which can be divided into two different subtypes.

Type 1 interferons include in particular the members of the interferon-α multigene family (there are about 14-20 different IFN-α molecules), IFN-τ (also called trophoblast IFN), and IFN-β and IFN-ω. The type I IFN genes are present as cluster on the short arm of chromosome 9.

Whereas IFN-α and IFN-ω) are preferentially formed by cells of the hematopoietic system, IFN-β is formed by non-hematopoietic cells, especially fibroblasts. The IFN-β is a glycoprotein (N-glycosylation), whereas most human IFN-α subtypes have no N-glycosylation. In the active form, IFN-α and IFN-β form dimers.

The huIFN-γ gene differs from the intron-free IFN type I genes by comprising three introns. IFN-γ belongs to the type II interferons. IFN-γ is a glycoprotein which is likewise a dimer in the active form. IFN-γ is formed in particular in CD4+ T-helper cells and in virtually all CD8+ cells. Despite a great functional similarity there is no substantial structural similarity between type I and type II interferons.

Interferons are important pharmaceuticals for the therapy of, for example, viral diseases, neoplastic diseases and immunodeficiencies. Systemic administration usually takes place intravenously, subcutaneously or intramuscularly. There are in addition local administration forms (e.g. intratumor injection and topical gel). Beside the lack of absorbability, oral use is also limited in the case of IFN-γ by the partial acid lability of the molecule.

Further cytokines include, in a nonlimiting manner, the colony-stimulating factor 4, heparin-binding neutrotrophic factor (HBNF), midkin (MD) and thymopoietin.

Monokines include according to the invention interleukin-1, tumor necrosis factors (e.g. TNF-α and -β), leucocyte-inhibiting factor (LIF) and the like.

Hematopoietic factors include according to the invention for example erythropoietin, granulocyte colony stimulating factor (G-CSF), granulocyte-macrophage stimulating factor (GM-CSF) and macrophage colony stimulating factor (M-CSF).

Anticoagulants include coagulation-modifying agents which circulate in the blood and control coagulation. Nonlimiting examples thereof are factor I, II, III, V, VI, VII, VIII, IX, X, XI and XII, α1-antitrypsin, α2-macroglobulin, antithrombin III, heparin cofactor II, kallikrein, plasmin, plasminogen, prokallikrein, protein C, protein S, thrombomodulin and the like.

Peptide hormones include for example insulin, glucagon, growth hormone, luteinizing hormone releasing hormone (LH-RH), adrenocorticotropin (ACTH), amylin, oxitocin, luteinizing hormone (LH), calcitonin, protein which controls the calcitonin gene, calcitonin N-terminal flanking peptide, somatotropin, somatostatin, somatomedin, tissue plasminogen activator (TPA), leuprolide acetate and the like.

Growth factors include according to the invention for example nerve growth factor (NGF), epidermal growth factor (EGF), fibroblast growth factor (FGF), insulin-like growth factor (IGF), transforming growth factor (TGF), platelet-derived growth factor (PDGF), hematocyte growth factor (HGF), growth hormone-releasing hormone (GHRH), human growth hormone (hGH) and the like.

Factors of the cardiovascular system are for example factors which regulate the blood pressure, arterosclerosis and the like, such as endothelins, endothelin inhibitors, endothelin antagonists, vasopressin (ADH), renin, angiotensin, atrial natriuretic factor (ANP) and the like.

Hormones derived from peptides include in a nonrestrictive manner activin, cholecystokinin (CCK), ciliary neurotrophic factor (CNTF), cortotropin-releasing factor (CRF or CRH), follicle-stimulating hormone (FSH), gastrin-inhibiting peptide (GIP), gastrin-releasing peptide, ghrelin, gonadotropin-releasing factor (GnRF or GNRH), growth hormone-releasing factor (GRF, GRH), human chorionic gonadotropin (hCH), inhibin A, inhibin B, leptin, lipotropin (LPH), α-melanocyte-stimulating hormone, β-melanocyte-stimulating hormone, γ-melanocyte-stimulating hormone, melatonin, motilin, pancreatic polypeptide, parathyroid hormones (PTH), placental prolactin, prolactin (PRL), prolactin-release-inhibiting factor (PIF), prolactin-releasing factor (PRF), thyrotropin (thyroid-stimulating hormone, TSH), thyroxine, triiodothyronine, vasoactive intestinal peptide (VIP) and the like.

Factors of the central or peripheral nervous system are for example opioid peptides (e.g. enkephalins, endorphins, kytorphins), neutrotrophic factor (NTF), tyroid hormone-releasing hormone (TRH), neurotensin and the like.

Endorphins or pharmacologically active derivatives thereof include in a nonlimiting manner dermorphin, dynorphin, α-endorphin, β-endorphin, γ-endorphin, σ-endorphin [Leu5]enkephalin, [Met5]enkephalin, substance P and the like.

Factors of the gastrointestinal system are for example secretin and gastrin.

Factors of the immune system are for example factors which control inflammations and neoplasms, and factors which attack infectious microorganisms, such as antibodies, chemotactic peptides or bradykinins.

An antibody may be a monoclonal antibody. In further embodiments, the antibody is a chimeric or humanized antibody, a fragment of a natural antibody or a synthetic antibody which can be produced by combinatorial techniques.

The antibodies described above and other binding molecules may be used for example for tissue identification. Antibodies may also be coupled to specific diagnostic substances for visualization of cells and tissues. They may moreover be coupled to therapeutically useable substances. Diagnostic substances include in a nonlimiting manner barium sulfate, iocetamic acid, iopanoic acid, calcium ipodate, sodium diatrizoate, meglumine diatrizoate, metrizamide, sodium tyropanoate and radiodiagnostic agents, including positron emitters such as fluorine-18 and carbon-11, gamma emitters such as iodine-123, technetium-99m, iodine-131 and indium-111, nuclides for nuclear magnetic resonance such as fluorine and gadolinium.

The term “therapeutically useable substance” means according to the invention any therapeutically useable molecule, including anticancer agents, compounds provided with radioactive iodine, technetium or further radioisotopes, toxins, cytostatic or cytolytic drugs, etc. Anticancer agents include for example aminoglutethimide, azathioprine, bleomycin sulfate, busulfan, carmustine, chlorambucil, cisplatin, cyclophosphamide, cyclosporin, cytarabine, dacarbazine, dactinomycin, daunorubin, doxorubicin, Taxol, etoposide, fluorouracil, interferon-α, lomustine, mercaptopurine, methotrexate, mitotane, procarbazine HCl, thioguanine, vinblastine sulfate and vincristine sulfate. Further anticancer agents are described for example in Goodman and Gilman, “The Pharmacological Basis of Therapeutics”, 8th edition, 1990, McGraw-Hill, Inc., especially chapter 52 (Antineoplastic Agents (Paul Calabresi and Bruce A. Chabner)). Toxins may be proteins such as pokeweed antiviral protein, cholera toxin, pertussis toxin, ricin, gelonin, abrin, diphtheria exotoxin or pseudomonas exotoxin. Toxin residues may also be high energy-emitting radionuclides such as cobalt-60.

In a further embodiment, the substance is a dermatological agent. Dermatological agents include cosmetics such as sunscreens which protect inner tissues of the skin (especially the tissues below the stratum corneum) from external factors such as UV rays in the UV-A and UV-B ranges (preferably radiation in the range from 280 to 400 nm) (e.g. p-aminobenzoic acid, p-dimethylaminobenzoic acid and their alkyl esters), agents for lightening the skin (e.g. hydroquinone), vitamins (e.g. vitamin A, C, D, E, K, nicotinic acid, thiamine, pyridoxine, vitamin B12, biotin, retinoids, flavonoids, pantothenate), provitamins, antioxidants, pigments, colorants and the like. Dermatological agents additionally include agents against pruritus and erythemas (e.g. hydrocortisone), against acne (e.g. erythromycin or tetracyclines), against herpes simplex (e.g. 5-iodo-2-deoxyuridine), against psoriasis or skin cancer (e.g. fluorouracil).

In a further embodiment, the agent which increases the absorption of a substance through the skin or mucosa is coupled to or loaded with a particle, preferably an optionally biodegradable nanoparticle, optionally biodegradable microparticle, optionally biodegradable nanobead, optionally biodegradable microbead, a capsule, emulsion, micelle, a liposome, a nonviral vector system or a viral vector system. The particle is preferably a particle derived from a virus (virus-like particle) which is preferably able to bind nonspecifically or in a targeted manner to cells and introduce a nucleic acid into them. The particle comprises a substance as described above, in particular a nucleic acid or peptide or protein which is to be absorbed by the skin or mucosa. Particles of these types are described for example in WO-A 00/46376. The particles include preferably: (a) a protein coat which preferably includes as fusion molecule a viral protein, an absorption-enhancing agent, preferably a peptide or protein, and where appropriate a heterologous cell-specific binding site, and (b) a nucleic acid which is present in the protein coat and has sequences for a virus-specific packaging signal and a structural gene. The term “virus” includes DNA viruses and RNA viruses, especially adenoviruses, adeno-associated viruses, vaccinia viruses, baculo viruses, hepatitis C viruses, hepatitis A viruses, influenza viruses and hepadna viruses. Examples of the latter are HBV, WHV (“woodchuck hepatitis virus”), GSHV (“ground squirrel hepatitis virus”), RBSHV (“red-bellied squirrel hepatitis virus”), DHV (“Pekin duck hepatitis virus”) and HHV (“heron hepatitis virus”), with HBV being preferred. The term “structural gene” includes any gene which codes for a polypeptide or protein such as the polypeptides and proteins described above.

In a preferred embodiment, the agent which increases the absorption of a substance through the skin or mucosa may be linked by absorption, noncovalent or covalent coupling, either directly or via a linker, to the particle, to the polymer(s) or monomer(s) which is/are used for the particle synthesis, or to other constituents of the particle.

In a preferred embodiment, the particle is loaded with a therapeutic, prophylactic or diagnostic substance, in which case the agent which increases the absorption of a substance through the skin or mucosa is linked to the particle or loaded therewith.

A particle of the invention can be produced by conventional methods.

Substances, in particular peptides or proteins, which are coupled according to the invention to an absorption-enhancing agent, in particular polypeptide or protein, can be used as immunogens in order to induce the production of antibody which preferably bind the immunogen immunospecifically.

The invention thus also relates to a method for producing antibodies, comprising an induction of antibody production through administration of substances, in particular peptides or proteins, which are coupled according to the invention to absorption-enhancing agents, to a creature, in particular a human or an animal, and an isolation of these antibodies.

### BRIEF DESCRIPTION OF THE DRAWINGS

FIG. 1 is a bar diagram which shows the amount of IFN-β detected in the serum 4 h and 8 h after oral administration of IFN-β-1a-TLM (TLM-1 and TLM-2). N1: negative control 1 (untreated animals); N2: negative control 2 (PreS1PreS2 in the feed); N3: negative control 3 (commercially available recombinant IFN-β-1a in the feed).

FIG. 2 is a bar diagram which shows the amount of IFN-β detected in the serum 4 h and 8 h after dermal administration of IFN-β-1a-TLM (TLM). N1: negative control 1 (untreated animals); N2: negative control 2 (dermal administration of commercially available recombinant IFN-β-1a).

FIG. 3 shows Western blot analyses for detecting PreS1PreS2-specific antibodies after oral administration of PreS1PreS2. Lane 1: cytochrome c; lane 2: PreS1PreS2; lane 3: heavy IgG chain.

FIG. 4 shows Western blot analyses for detecting PreS1PreS2 in the serum after dermal administration of PreS1PreS2. Lane 1: positive control; lanes 2 to 5: negative controls (untreated animals); lanes 6 to 9: sera from animals treated with PreS1PreS2.

### DETAILED DESCRIPTION OF THE INVENTION

The term “absorption” means according to the invention the uptake of substances from the surface of the body. The absorption includes in particular absorption through the skin (i.e. transdermal, percutaneous) or through mucosa (mucous membrane) (i.e. transmucosal) preferably into the blood stream, lymphatic system and/or lower layers of skin, from where distribution throughout the body is possible. The absorption may take place by the passive mechanism of diffusion or else by active transport mechanisms.

In an absorption via the skin or mucosa of a patient, a substance which is coupled to an absorption-enhancing agent preferably enters the outermost layer of the skin (stratum corneum) according to the invention. In a preferred embodiment, the substance coupled to the absorption-enhancing agent reaches the underlying layers. In a further preferred embodiment, the substance coupled to the absorption-enhancing agent is released into the blood stream.

The term “increasing” relates to an elevation, enhancement or improvement compared with a previous state. Thus, for example, the term “increase in the absorption” relates to an elevation of absorption, i.e. a larger amount of a substance is absorbed in a particular time, especially through an increase in the rate at which a substance penetrates through a body barrier such as skin and mucous membranes.

This may relate to the case where a substance was not originally able to be absorbed, and the substance is, after the “increase in the absorption”, able to be absorbed. This may also relate to a case where a substance was originally already able to be absorbed but the ability of the substance to be absorbed is enhanced after the “increase in the absorption”.

The term “substance which is poorly absorbed” means that the substance is absorbed only slightly or not at all, and in particular does not provide a therapeutically effective concentration with a usual dose quantity.

The terms “increasing the bioavailability” and “increasing the permeability” are to be interpreted in a corresponding manner.

The term “bioavailability” characterizes the rate and the extent of release and absorption, and availability at the site of action, of the therapeutically effective portion of a medicament from the particular pharmaceutical forms. It can be determined by measuring the drug concentration in the body fluids and the acute pharmacological effect.

The term “permeability” relates to the property, e.g. of skin and mucous membranes, of allowing a substance to pass through. The terms “permeation ability” and “penetration ability” relate to the ability of a substance to pass through such a barrier.

“Transdermal or transmucosal product” refers according to the invention to a substance, in particular an active pharmaceutical ingredient, which was originally absorbed poorly or not at all by skin or mucosa but has been modified so that it is absorbed by the skin or mucosa and is therefore suitable for administration onto the skin or mucosa.

“Mucosa” or “mucous membrane” may be according to the invention any mucous membrane of a mammal, including humans.

Examples of mucous membranes include according to the invention the mucous membrane of the gastrointestinal tract (e.g. intestinal mucosa, gastric mucosa), eye mucosa, nasal mucosa, tracheal/bronchial/lung mucosa, mucous membrane of the oral cavity, of the rectum, of the genital tract, of the vagina, of the ureter and the like.

The mucous membrane is preferably a mucous membrane of the nose, of the mouth or of the gastrointestinal tract.

“Transdermal administration” or “transmucosal administration” means provision via the skin or mucosa.

“Agents which increase the absorption of a substance”, “absorption enhancers” or “absorption-enhancing agents” for the purposes of the present invention are substances or products which promote the transport of other substances through barriers and constrictions, especially permeation barriers, and preferably increase their bioavailability, ability to be absorbed and/or permeation ability (penetration ability). The permeation barriers include in particular human and animal skin layers, especially dermis (especially stratum corneum) and mucosa. The agent which increases the absorption of a substance through the skin or mucosa is preferably free of toxic side effects.

Methods for the covalent or noncovalent linkage (coupling) of two or more reagents are known to a skilled worker.

“Noncovalent” linkages include in a nonlimiting manner ionic interactions, hydrogen bonds, van der Waal's interactions (hydrophobic interactions) and linkages resulting from inclusion of one compound inside another (e.g. in crown ethers and cage compounds).

Covalent coupling of, for example, peptides and proteins is possible with use of coupling agents such as N,N′-dicyclohexylcarbodiimide (DCC) or N,N′-diisopropylcarbodiimide (DIPCDI) or by recombinant techniques in a manner known per se. Suitable synthetic methods are described for example in “The Peptides: Analysis, Structure”, Biology, volume 1: “Methods of Peptide Bond Formation”, Gross and Meienhofer (editors), Academic Press, New York (1979) and Izumiya et al., “Synthesis of Peptides”, Maruzen Publishing Co., Ltd., (1975).

A nucleic acid is preferably according to the invention deoxyribonucleic acid (DNA) or ribonucleic acid (RNA). Nucleic acids include according to the invention genomic DNA, cDNA, mRNA, rRNA, tRNA, recombinantly produced and chemically synthesized molecules. A nucleic acid may according to the invention be in the form of a single-stranded or double-stranded and linear or covalently circularized molecule.

“Derivative” of a nucleic acid means according to the invention that single or multiple nucleotide substitutions, deletions and/or additions are present in the nucleic acid. The term “derivative” also includes a chemical derivatization of a nucleic acid on a base, a sugar or phosphate of a nucleotide. The term “derivative” also includes nucleic acids which comprise non-naturally occurring nucleotides and nucleotide analogues.

The nucleic acids described according to the invention are preferably isolated. The term “isolated nucleic acid” means according to the invention that the nucleic acid (i) has been amplified in vitro, for example by polymerase chain reaction (PCR), (ii) has been produced recombinantly by cloning, (iii) has been purified, for example by cleavage and fractionation by gel electrophoresis, or (iv) has been synthesized, for example by chemical synthesis. An isolated nucleic acid is a nucleic acid which is available for manipulation by recombinant DNA techniques.

The term “expression” is used according to the invention in its most general meaning and includes the production of RNA or of RNA and protein. It also includes partial expression of nucleic acids. The expression may moreover take place transiently or stably.

The term “sequence derived from an amino acid sequence” relates according to the invention to derivatives of the latter sequence.

“Derivatives” of a protein or polypeptide or of an amino acid sequence for the purposes of this invention include amino acid insertion variants, amino acid deletion variants and/or amino acid substitution variants.

Amino acid insertion variants include amino- and/or carboxy-terminal fusions, and insertions of one or more amino acids in a particular amino acid sequence. Amino acid sequence variants with an insertion have one or more amino acid residues introduced into a predetermined site in an amino acid sequence, although random insertion with suitable screening of the resulting product is also possible. Amino acid deletion variants are characterized by the removal of one or more amino acids from the sequence. Amino acid substitution variants are distinguished by removal of at least one residue in the sequence and insertion of another residue in its place. The modifications are preferably present at positions in the amino acid sequence which are not conserved between homologous proteins or polypeptides. Amino acids are preferably replaced by others having similar properties such as hydrophobicity, hydrophilicity, electronegativity, volume of the side chain and the like (conservative substitution). Conservative substitutions relate in this connection for example to replacement of one amino acid by another, with both amino acids being listed in the same group below:
1. small aliphatic, nonpolar or slightly polar residues: Ala, Ser, Thr (Pro, Gly)
2. negatively charged residues and their amides: Asn, Asp, Glu, Gln
3. positively charged residues: His, Arg, Lys
4. large aliphatic, nonpolar residues: Met, Leu, Ile, Val (Cys).
5. large aromatic residues: Phe, Tyr, Trp.

Three residues are placed in parentheses because of their particular importance for the protein architecture. Gly is the only residue without a side chain and thus confers flexibility on the chain. Pro has an unusual geometry which greatly restricts the chain. Cys can form a disulfide bridge.

The amino acid variants described above can easily be prepared by means of known peptide synthesis techniques such as, for example, by solid phase synthesis (Merrifield, 1964) and similar methods or by recombinant DNA manipulation. Techniques for introducing substitution mutations at predetermined sites in DNA having a known or partially known sequence are well known and include, for example, M13 mutagenesis. Manipulation of DNA sequences to produce proteins with substitutions, insertions or deletions and the general recombinant methods for expression of proteins for example in a biological system (such as mammalian, insect, plant and viral systems) are described in detail for example in Sambrook et al. (1989).

“Derivatives” of proteins or polypeptides also include according to the invention single or multiple substitutions, deletions and/or additions of any molecules which are associated with the enzyme, such as carbohydrates, lipids and/or proteins or polypeptides.

In one embodiment, “derivatives” of proteins or polypeptides include the modified analogues which result from glycosylation, acetylation, phosphorylation, amidation, palmitoylation, myristolylation, isoprenylation, lipidation, alkylation, derivatization, introduction of protective/blocking groups, proteolytic cleavage or linkage to an antibody or to another cellular ligand. Derivatives of proteins or polypeptides may also be prepared by other methods such as, for example, by chemical cleavage with cyanogen bromide, trypsin, chymotrypsin, papain, V8 protease, NaBH₂, acetylation, formylation, oxidation, reduction or by metabolic synthesis in the presence of tunicamycin.

The term “derivative” also extends to all functional chemical equivalents of the proteins or polypeptides.

A part or fragment of a polypeptide or protein displays according to the invention a functional property of the polypeptide or protein from which it is derived. Such functional properties include interaction with other molecules such as antibodies, polypeptides or proteins, selective binding of nucleic acids and enzymatic activity. A part or fragment of a peptide or protein preferably includes according to the invention a sequence of at least 6, in particular at least 8, at least 10, at least 12, at least 15, at least 20, at least 30 or at least 50 consecutive amino acids from the peptide or protein.

The terms “active pharmaceutical ingredient”, “pharmaceutically active substance” or “pharmaceutically active” relate according to the invention to any agent which can be employed in therapy (including prophylaxis) or diagnosis. The agent is in particular any therapeutic or prophylactic agent which can be employed for the treatment (including prevention, alleviation or curing) of a disease, of symptoms or of an injury of a patient and has the desired biological or pharmacological effect.

An active pharmaceutical ingredient may be a “dermally acting dermatological active ingredient” or a “systemically acting dermatological active ingredient”. The term “dermally acting dermatological active ingredient” as used herein relates to the chemical and biochemical substances which, when applied to the skin of a patient, elicit a beneficial topical effect which may be cosmetic in nature or therapeutic in nature (e.g. a moderation of a skin disorder). The term “systemically acting dermatological active ingredient” as used herein relates to the chemical and biochemical substances which, when applied to the skin of a patient, enter the bloodstream and show a therapeutic effect. The terms “dermally acting dermatological active ingredient” and “systemically acting dermatological active ingredient” are not intended to be mutually exclusive because a number of active pharmaceutical ingredients have both dermal and systemic activity. An active pharmaceutical ingredient may also be a “mucosally acting mucosal active ingredient” or a “systemically acting mucosal active ingredient”, where the terms “mucosally acting mucosal active ingredient” and “systemically acting mucosal active ingredient” have a meaning corresponding to the previously defined terms “dermally acting dermatological active ingredient” and “systemically acting dermatological active ingredient”, respectively.

The active pharmaceutical ingredient is preferably formulated in neutral or salt form. Pharmaceutically acceptable salts include in a nonlimiting manner those formed with free amino or carboxyl groups. Suitable acids for preparing acid addition salts are inorganic acids such as HCl, HBr, H₂SO₄, HNO₃, H₃PO₄ and the like, and organic acids such as acetic acid, propionic acid, oxalic acid, maleic acid, malonic acid, succinic acid, malic acid, fumaric acid, tartaric acid, citric acid, benzoic acid, cinnamic acid, mandelic acid, methane-sulfonic acid, p-toluenesulfonic acid, salicylic acid and the like. Basic compounds able to form salts with carboxyl groups include in a nonlimiting manner NaOH, KOH, NH₃, Ca(OH)₂, iron hydroxide, isopropylamine, triethylamine, 2-ethylaminoethanol, histidine, procaine and the like.

The active pharmaceutical ingredient may also be a medicament precursor which can be activated before, during or after penetration of the active ingredient through the skin or mucosa.

The term “medicament precursor” relates to an agent which is inactive but can be converted into an active form by enzymatic, chemical or physical activation.

Pharmaceutical compositions can be produced in a manner known per se and usually comprise suitable pharmaceutically acceptable excipients and carriers.

The term “pharmaceutically acceptable” relates to a substance which causes no or only a slight significant irritation or toxicity in the treated patient and does not abolish the biological activity and properties of the active ingredient or interacts therewith.

The term “carrier” relates according to the invention to one or more compatible solid or liquid fillers, diluents, adjuvants, excipients or capsule substances which are suitable for administration to a person. The term “carrier” relates to an organic or inorganic ingredient which is natural or synthetic in nature and in which the active ingredient is combined in order to facilitate use. The ingredients of the pharmaceutical composition of the invention are usually such that no interaction which substantially impairs the desired pharmaceutical activity occurs.

The carriers are preferably sterile liquids such as water or oils, including those derived from petroleum, animals or plants, or of synthetic origin, such as, for example, peanut oil, soybean oil, mineral oil, sesame oil, sunflower oil and the like. Salt solutions and aqueous dextrose and glycerol solutions can also be used as aqueous carriers.

Examples of excipients and carriers are acrylic and methacrylic derivatives, alginic acid, sorbic acid derivatives such as α-octadecyl-ω-hydroxypoly(oxy-ethylene)-5-sorbic acid, amino acids and derivatives thereof, especially amine compounds such as choline, lecithin and phosphatidylcholine, gum arabic, aromatizing substances, ascorbic acid, carbonates such as, for example, sodium, potassium, magnesium and calcium carbonate and bicarbonate, hydrogen phosphates and phosphates of sodium, potassium, calcium and magnesium, carmellose sodium, dimeticone, colors, flavorings, buffer substances, preservatives, thickeners, plasticizers, gelatin, glucose syrups, malt, colloidal silicon dioxide, hydromellose, benzoates, especially sodium and potassium benzoate, macrogol, skim milk powder, magnesium oxide, fatty acids and derivatives and salts thereof such as stearic acid and stearates, especially magnesium and calcium stearate, fatty acid esters and mono- and diglycerides of edible fatty acids, natural and synthetic waxes such as beeswax, yellow wax and Montan glycol wax, chlorides, especially sodium chloride, polyvidone, polyethylene glycols, polyvinylpyrrolidone, povidone, oils such as castor oil, soybean oil, coconut oil, palm kernel oil, sugars and sugar derivatives, especially mono- and disaccharides such as glucose, fructose, mannose, galactose, lactose, maltose, xylose, sucrose, dextrose and cellulose and derivatives thereof, shellac, starch and starch derivatives, especially corn starch, tallow, talc, titanium dioxide, tartaric acid, sugar alcohols such as glycerol, mannitol, sorbitol and xylitol and derivatives thereof, glycol, ethanol and mixtures thereof.

The pharmaceutical compositions may preferably also comprise in addition wetting agents, emulsifiers and/or pH-buffering agents.

In a further embodiment, the pharmaceutical compositions may comprise an additional absorption enhancer. These additional absorption enhancers may, if desired, replace an equimolar amount of the carrier in the composition. Examples of such additional absorption enhancers include in a nonrestrictive manner eucalyptol, N,N-diethyl-m-toluamide, polyoxyalkylene alcohols (such as propylene glycol and polyethylene glycol), N-methyl-2-pyrrolidone, isopropyl myristate, dimethylformamide (DMF), dimethyl sulfoxide (DMSO), dimethylacetamide (DMA), urea, diethanolamine, triethanolamine and the like (see, for example, Percutaneous Penetration Enhancers, edited by Smith et al. (CRC Press, 1995)). The amount of additional absorption enhancer in the composition may depend on the desired effects to be achieved.

Since a large number of proteolytic enzymes is present in the mucosa and its surroundings, a protease inhibitor may be incorporated into the composition of the invention in order to prevent degradation of a peptide or protein active ingredient and thus to increase the bioavailability. Examples of protease inhibitors include in a nonrestrictive manner aprotinin, leupepsin, pepstatin, α2-macroglobulin and trypsin inhibitor. These inhibitors can be used alone or in combination.

The pharmaceutical compositions of the invention may be provided with one or more coatings. The solid oral dosage forms are preferably provided with a gastro-resistant coating or are in the form of a gastro-resistant, hardened soft gelatin capsule.

The dosage forms may include materials which release the pharmaceutically active substance in a specific segment of the gastrointestinal tract, thus enhancing site-directed provision.

The compositions described herein may also be administered as formulation with delayed release (i.e. a formulation which brings about slow release of the medicament after administration). Such formulations with delayed release are known.

The pharmaceutical compositions may be formulated according to the invention for administration by any transdermal or transmucosal route, including, for example, for topical, oral, enteral, intracranial, sublingual, nasal, buccal, vaginal, ocular or urethral administration. Particular preference is given to enteral, and even more preference to oral dosage forms, especially gastro-resistant formulations and slow-release formulations of oral forms. However, rectal pharmaceutical forms such as suppositories, vaginal pharmaceutical forms such as suppositories, and nasally applicable preparations such as nasal sprays are also possible.

In a preferred embodiment, the pharmaceutical composition is incorporated into the matrix of a patch in order to deliver the substance, especially the active pharmaceutical ingredient, which is coupled to the absorption-enhancing agent to the skin over a prolonged period.

The pharmaceutical formulations are for example in the form of tablets, suppositories, pastilles, coated tablets, drops, solutions, suspensions, emulsions (preferably oil-in-water or water-in-oil emulsions), ointments, gels, pastes, films, juices, syrups, nasal sprays, vaginal suppositories or tablets, capsules, granules, pellets, microtablets, powders, rectal suppositories, rectal capsules, aerosols, shampoos or sprays. Particular preference is given to hard or soft gelatin capsules, where appropriate with gastro-resistant coating, with very particular preference for hardened soft gelatin capsules.

The pharmaceutical composition may according to the invention be an indirect dose form such as an oral formulation for administration onto the gastric or intestinal mucous membranes. However, the composition may also be administered directly onto a mucous membrane.

The pharmaceutical compositions are preferably according to the invention medicaments which can be administered topically or orally.

The term “patient” means according to the invention a human, non-human primate or another animal, especially mammal such as cow, horse, pig, sheep, goat, dog, cat, bird such as chicken or rodent such as mouse and rat. In a particularly preferred embodiment, the patient is a human.

The pharmaceutical compositions of the invention are preferably sterile and are administered in effective amounts. An “effective amount” relates to the amount which achieves, alone or together with further doses, a desired response or a desired physiological effect. In the case of treatment of a particular disorder or of a particular condition, the desired response relates to inhibiting the progress of the disease. This includes a slowing of the progression of the disorder and in particular a stoppage of the progression of the disorder. The desired response on treatment of a disease or of a condition may also be a delay in the onset or a prevention of the onset of the disease or of the condition.

The effective amount can be selected according to the activity of the specific active pharmaceutical ingredient and its therapeutically effective dose. However, it is preferred to incorporate a somewhat larger amount than the desired dose, because the bioavailability of any active substance can never be 100%, i.e. the administered dose is not completely absorbed. For example, physiologically active peptides or proteins are degraded by digestive juices in the gastrointestinal tract or hydrolyzed by enzymes in the gastrointestinal tract. An effective amount of a pharmaceutical composition will also depend on factors such as the patient's condition to be treated, the severity of the disorder, the individual patient's parameters, including age, physiological condition, height and weight, the duration of treatment, the nature of a concomitant therapy (if present), the specific administration route, the desired administration period and similar factors.

In the case where a patient's response is inadequate with an initial dose, it is possible to employ higher doses (or effectively higher doses which are achieved by a different, more localized administration route).

An alternative possibility is for higher doses to be achieved by increasing the amount of absorption-enhancing agent, the concentration of the substance (especially of the active pharmaceutical ingredient) and/or the amount of additional absorption enhancer in the formulation, enlarging the area to which the formulation is applied, or by a combination thereof.

The present invention is described in detail by the following examples and figures, which serve exclusively for illustration and are not to be understood as limiting. Further embodiments which do not extend beyond the scope of the invention and the scope of the appended claims are accessible to the skilled worker on the basis of the description and the examples.

### EXAMPLES

### Example 1

### Preparation and Use of Protein Expression Constructs

a. Cloning

pQe8 expression vectors which coded for IFN-β fused to the sequence P-L-S-S-I-F-S-R-I-G-D-P (TLM) at the 5′ or 3′ end were prepared. The corresponding constructs without TLM were prepared for control experiments. The identity of these constructs was verified by sequencing.

Starting from the construct pCI-eIFNb.mv, which comprises an huIFN-β-specific cDNA, PCR was used to amplify cDNAs which code for IFN-β-specific fusion proteins which include the TLM sequence at the N or C terminus. The forward primers had a BamHI-specific cleavage site at their 5′ end and a HindIII-specific cleavage site at the 3′ end. The following primers were specifically used:

| | |
|---|---|
| A) | ggg aag ctt tca agg gtc ccc aat cct cga gaa gat |
| | tga cga taa ggg gtt tcg gag gta acc tgt aag |
| B) | ggg aag ctt tca gtt tcg gag gta acc tgt |
| C) | ggg gga tcc atg agc tac aac ttg ctt gga |
| D) | ggg gga tcc ccc tta tcg tca atc ttc tcg agg att |
| | ggg gac cct atg agc tac aac ttg ctt gga |

With the D/B primer combination, a cDNA which comprises the sequence coding for TLM at the 5′ end was amplified. With the C/A primer combination, a sequence which comprises the TLM-specific sequence at the 3′ end was amplified. For control experiments, the IFN-β-specific cDNA without 5′- or 3′-specific extensions was amplified with the C/B primer combination.

The respective PCR products were purified using PCR purification spin columns in accordance with the manufacturer's (Quiagen) instructions, BamHI/HindIII-cleaved and again purified. The fragments restricted in this way were ligated into the BamHI/HindIII-cleaved and dephosphorylated bacterial expression vector pQe8 (Quiagen). The vector pQe8 comprises the sequence coding for an amino-terminal hexa-His tag, so that all the IFN-β-specific proteins were formed as hexa-His fusion proteins.

The ligation mixture was used to transform competent bacteria (DH5α). The Amp resistance encoded on the plasmid pQe8 allowed selection on Amp-containing media.

Plasmid DNA was isolated from clones growing under these conditions and was analyzed by BamHI/HindIII restriction. Positive clones were then characterized by sequencing.

b. Expression

Induction of the formation of IFN-β-specific fusion proteins took place as follows: 900 ml of Amp-containing LB medium (cₐₘₚ=100 mg/l) were inoculated with 100 ml of a preculture grown until stationary, and expanded at 37° C. until the OD₆₀₀ was 0.8. Gene expression was induced by adding IPTG to a final concentration of 1 mM (expression of genes inserted into pQe8 takes place under the control of the lac repressor). Harvesting took place 2-3 h after starting induction.

### Example 2

### Protein Isolation

The bacterial pellet which had been washed twice in PBS was resuspended in 50 mM NaH₂PO₄/300 mM NaCl/8 mM imidazole, pH 8.0 (native purification), and the bacteria were disrupted with ultrasound. Non-disrupted bacteria, and bacterial detritus, were sedimented by centrifugation. The supernatant was loaded onto an Ni-NTA-agarose column equilibrated with 50 mM NaH₂PO₄/300 mM NaCl/8 mM imidazole, pH 8.0 (Ni-NTA-agarose enables hexa-His-tagged proteins to be purified by affinity chromatography). The column was loaded at a flow rate of 1 ml/min.

After the loading of the column and the washing out of unbound proteins, a buffer with 50 mM NaH₂PO₄/300 mM NaCl/20 mM imidazole, pH 8.0, was used to elute weakly bound proteins. The specifically bound hexa-His-tagged IFN-β fusion proteins were eluted by a linear gradient between a buffer with 50 mM NaH₂PO₄/300 mM NaCl/20 mM imidazole, pH 8.0 and a buffer with 50 mM NaH₂PO₄/300 mM NaCl/250 mM imidazole, pH 8.0. Eluted proteins were detected by simultaneous detection of the absorption at 215, 260 and 280 nm. The eluate was collected in 1 ml fractions.

The isolation took place with use of an AEKTA explorer or AEKTA purifier system.

For further purification, in a few cases a reversed phase chromatography was carried out using an RP18 column. For this purpose, the eluate from the Ni-NTA column was diluted 1:5 with the running buffer of the RP column (0.1% TFA in H₂O) and loaded onto the column. Elution took place with a linear gradient between 0.1/TFA in H₂O and 80% acetonitrile/H₂O.

Analysis of the Proteins:

The purity of the proteins isolated in this way was analyzed by Laemmli SDS-PAGE. The gels were Coomassie-stained or subjected to a silver stain (Heukeshoven/Dernick method).

The identity of the detected protein bands with IFN-β (IFN-β-1b) was demonstrated by Western blottings. The proteins were transferred to a PVDF membrane by means of electroblotting by the semi-dry method (Kyshe/Andersen). The transferred IFN-β-specific protein was labelled using an IFN-β-specific sheep serum. Detection took place by fluorography by means of peroxidase-conjugated secondary antibody using the ECL system (Amersham).

It was possible in this way to isolate IFN-β-1b and TLM-IFN-β-1b in more than 95% purity. The yield was about 400-700 μg per liter.

TLM-IFN-β-1b of more than 98% purity could be isolated by reversed phase chromatography.

### Example 3

### Demonstration of Cell Permeability

a. Cell Fractionation

The human hepatoma cell line huH7 was incubated in the presence of 0.5 μM IFN-β-1b-specific proteins in medium for 30 min. Surface-bound IFNs were removed by washing the cells, after removal of the medium, with Na₂CO₃/NaHCO₃ buffer, pH 9.5, for 5 sec and then in PBS. After the cells had been scraped off they were lyzed under mild conditions using a Potter homogenizer. After removal of unlyzed cells and the cell nuclei by centrifugation at 13 000 rpm in an Eppendorf centrifuge for 30 sec, the lysate was subjected to a differential centrifugation. It was possible by ultracentrifugation at 100 000 rpm (430 000 g) for 18 min to isolate the cytosol and the microsomal fraction. The cell fractions isolated in this way were subjected to an SDS-PAGE and then analyzed by Western blottings using the IFN-β-specific serum.

The Western blotting analysis of the subcellular fractionation showed that only TLM-IFN-β-1b, but not wt-IFN, is detectable in the cytosol. Detection of extracellularly added TLM-IFN-β-1b in the cytosol confirms the cell permeability and underlines the fact that uptake did not take place by an endosome-associated route.

b. Immunofluorescence Microscopy

The human hepatoma cell line huH7 and COS cells (hamster) were incubated in the presence of 0.5 μM IFN-β-1b-specific proteins in medium for 30 min. Surface-bound IFNs were removed by washing the cells, after removal of the medium, with Na₂CO₃/NaHCO₃ buffer, pH 9.5, for 5 sec and then in PBS. The washed cells were fixed in ice-cold ethanol/DAPI (to stain the cell nucleus) for 10 min. The fixation was followed by rehydration in PBST for 30 min. 10% BSA was used to block nonspecific binding sites. huIFN-β-specific sheep serum was used to label the IFN-β. A Cy3-coupled secondary antibody was used for detection. A Leica fluorescence microscope was used for the evaluation.

The immunofluorescence microscopy showed that, unlike wtIFN which gave only a very weak background signal, TLM-IFN-β-1b is readily detectable in the huH7 and in the COS cells. It is detectable in virtually all cells. TLM-IFN-β-1b is homogeneously distributed over the cell, and no specific accumulation in individual subcellular compartments is to be observed.

### Example 4

### Demonstration of Oral Availability by Feeding Tests

B6 mice were kept without feed overnight. The following morning, the animals received a weighed feed brick which was impregnated with IFN-β-1b-specific protein solution. Weighing of the brick after the end of the feeding test allowed the quantitative oral intake of IFN to be estimated. The animals were sacrificed with CO₂ and the blood was removed as EDTA blood by cardiac puncture. After removal of cellular constituents, the serum was analyzed by Western blotting and an huIFN-β-specific ELISA.

The Elisa values were adjusted for the quantitative IFN-β-1b intake (amount of feed) and related to the c/o value. The c/o value was set at 1.

The following values were found for the animals fed with TLM-IFN-β-1b (animals 1-4) and the animals which received wtIFN (animals 5-7):

| | |
|---|---|
| TLM-IFN-β-1b | wt IFN |

| No. of the | | | | | | | |
|---|---|---|---|---|---|---|---|
| animal | 1 | 2 | 3 | 4 | 5 | 6 | 7 |
| Elisa value | 1.8 | 1.4 | 2.1 | 1.9 | 0.4 | 0.3 | 0.4 |

These results show that orally administered TLM-IFN-β-1b was clearly detectable in the serum, whereas orally administered wtIFN was detected only in small amounts.

### Example 5

### Preparation and Use of IFN-β-1a-TLM Using a Eukaryotic IFN-β-TLM-specific Expression Vector

a) Cloning

Starting from the construct pCI-eIFNb.mv, which comprises a human IFN-β (huIFN-β)-specific cDNA, PCR was used to amplify the cDNA coding for IFN-β-specific fusion proteins. This cDNA codes for a complete IFN-β-specific fusion protein which includes the cell permeability-conferring TLM-encoding sequence at the C terminus in the open reading frame. The primers were designed so that the amplicon had a BamHI-specific cleavage site in each case at its 5′ end and 3′ end.

The PCR product was purified using PCR purification spin columns according to the manufacturer's (Quiagen) instructions, BamHI-cleaved and again purified. The fragments restricted in this way were ligated into the BamHI-cleaved and dephosphorylated eukaryotic expression vector pcDNA.3.1 (Invitrogen). The ligation mixture was used to transform competent bacteria (DH5α). The Amp resistance encoded on the plasmid allowed selection on Amp-containing media. Plasmid DNA was isolated from the clones grown under these conditions and was initially analyzed by BamHI restriction. Positive clones were then characterized by sequencing and their orientation was checked.

b. Expression and Purification

The formation of IFN-β-specific fusion proteins in which IFN-β was glycosylated as in the native protein (IFN-β-1a) took place as described below:

30 bottles (T175) of huH7 cells at 70% confluence were transiently transfected with 6 μg of pCIFNbTLM using Lipofectin. The transfection took place in accordance with the manufacturer's (DOTAP, Roche) instructions. 48 h after the medium change, the medium was collected and the IFN-β-1a-TLM produced was enriched by fractional ammonium sulfate precipitation (20% ammonium sulfate saturation followed by 70% ammonium sulfate saturation). The precipitate was resuspended in PBS and dialyzed against PBS for 12 to 18 h in order to remove the excess ammonium sulfate. This was followed by preparative gel filtration using a calibrated Superdex 75 column. The fractions identified as IFN-β-positive by Western blot analysis using an huIFN-β-specific anti-serum were combined and further purified on a MonoQ ion exchanger column. Elution took place by a linear gradient from 20 to 1000 mM NaCl, buffered in 40 mM Tris with a pH of 7.5 and 2% ethanol. As was found by Western blot analysis, silver-stained SDS gels and analytical HPLC, it was possible in this way to isolate IFN-β-1a-TLM in a purity of more than 90%.

Demonstration of the functionality took place:

by measuring the antiviral activity. For this purpose, HepG2.2.15 cells (a stably HBV-producing cell line) were incubated in the presence of various amounts of IFN-β-1a-TLM. It was possible by taqman PCR to observe a regression in virus production by a factor of 1000 (see also example 8).
by measuring the induction of 2′,5′-oligoadenylate synthetase by means of a specific RIA. This assay is based on the fact that IFN-β can bind to cells not infected with a virus and thus induces the formation inter alia of 2′,5′-oligoadenylate synthetase, which leads to a degradation of viral RNA (cf., for example, Takane et al., Jpn. J. Pharmacol. 90, 304-312, 2002).

### Example 6

### Demonstration of the Oral Availability of IFN-β-1a-TLM by Feeding Tests

B6 mice were kept without feed for 18 h. At the start of the test, the animals received a weighed piece of toasted bread (about 3.5 to 4.5 g) which contained 10⁴ U of IFN-β-1a-TLM from example 5 (TLM). The animals used as negative controls had been subjected to no treatment (N1), or had received feed with 1 ml of a 200 μM PreS1PreS2 solution (negative control N2) or feed with 10⁴ U of commercially available recombinant IFN-β-1a (negative control N3). The animals were fed for 4 or 8 h. Two separate experiments were carried out for all treatment protocols (TLM-1, TLM-2, N1-1, N1-2, etc.). The animals were sacrificed with CO₂ and the blood was removed as EDTA blood by cardiac puncture. After removal of cellular constituents, the serum was analyzed using a commercial huIFN-β-specific ELISA. Various amounts of commercially available recombinant IFN-β-1a (krIFN-β-1a) were measured for a calibration plot. Tables 1 to 3 below represent the resulting measurements:

TABLE 1
Measurements for the calibration plots

| krIFN-β-1A (I.U.) | Exp. 1 | Exp. 2 |
|---|---|---|
| 2.5 | 0.126 | 0.286 |
| 5 | 0.172 | 0.353 |
| 10 | 0.353 | 0.540 |
| 20 | 0.606 | 0.758 |
| 50 | 1.274 | 1.187 |
| 100 | 1.749 | 1.705 |
| 200 | 1.879 | 1.750 |

TABLE 2
Measurements, means and calculated amounts
after oral administration of IFN-β-1a-TLM

| TLM | |
|---|---|
| 4 h | 8 h |

| | TLM-1 | TLM-2 | TLM-1 | TLM-2 |
|---|---|---|---|---|
| | 0.387 | 0.498 | 0.739 | 0.629 |
| | 0.030^{#} | 1.175 | 0.575 | 0.475 |
| | 0.418 | 1.781 | 0.554 | 0.766 |
| | 1.289^{*} | 1.244 | 1.411^{*} | 0.670 |
| | 1.420^{*} | 1.270 | 0.717 | 1.112 |
| | 1.287^{*} | 1.778 | 0.306 | 1.007 |
| | 1.215^{*} | 1.527 | 0.191 | 0.839 |
| | 1.547^{*} | — | 1.000^{*} | 0.601 |
| | 1.285^{*} | — | 0.163 | 0.632 |
| Mean | 1.106 | 1.325 | 0.628 | 0.748 |
| I.U. | 41 | 54 | 20 | 22 |

^{#}was not included in the calculation of the mean because food was refused
^{*}was assayed for IFN-β-activity (see example 8)

TABLE 3
Measurements, means and calculated amounts for
the negative controls

| | N1 | | N2 | | N3 | | | |
|---|---|---|---|---|---|---|---|---|
| | 8 h | | 8 h | | 4 h | | 8 h | |
| | N1-1 | N1-2 | N2-1 | N2-2 | N3-1 | N3-2 | N3-1 | N3-2 |
| | 0.041 | 0.005 | 0.034 | 0.008 | 0.104 | 0.011 | 0.091 | 0.109 |
| | 0.086 | 0.003 | 0.051 | 0.007 | 0.044 | 0.005 | 0.049 | 0.025 |
| | 0.047 | 0.006 | 0.107 | 0.007 | 0.034 | 0.026 | 0.032 | 0.009 |
| | 0.032 | 0.002 | 0.061 | 0.014 | 0.091 | 0.060 | 0.078 | 0.013 |
| | 0.035 | 0.000 | 0.066 | 0.009 | 0.072 | 0.005 | 0.077 | 0.004 |
| | | | 0.083 | 0.050 | 0.072 | 0.249 | | |
| | | | 0.083 | 0.005 | 0.121 | 0.005 | | |
| | | | 0.130 | 0.006 | 0.131 | 0.010 | | |
| | | | 0.057 | 0.123 | | | | |
| Mean | 0.048 | 0.003 | 0.075 | 0.025 | 0.084 | 0.046 | 0.065 | 0.032 |
| I.U. | 1 | 0 | 2 | 0 | 2 | 0 | 2 | 0 |

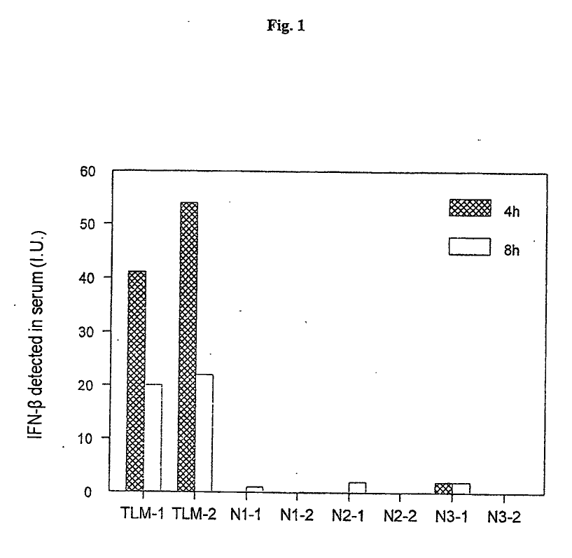

The ELISA values were averaged for each experiment and the amount detected in the serum was calculated using the calibration plot. In FIG. 1, the calculated amounts (I.U.) of the IFN-β detected in the serum are plotted in a bar diagram. FIG. 1 shows that the amount of IFN-β in the serum was distinctly raised after oral administration of IFN-β-1a-TLM for 4 h and 8 h, respectively, with the amount after 4 h being about twice as high as the amount after 8 h. In contrast thereto, no significant increase in the amount of IFN-β in the serum was detectable in any negative control. Consequently, the results show that a distinct increase was possible in the absorption of IFN-β via the mucous membrane through the coupling of TLM to IFN-β.

### Example 7

### Demonstration of the Dermal Availability of IFN-β-1a-TLM

B6 mice were carefully shaved in order not to injure the skin and kept with a gauze dressing (2×6 cm, 2-layer) which was impermeable on the outside and had been impregnated in 10⁴ U of IFN-β-1a-TLM from example 5 for 4 and 8 h (TLM). Animals which were subjected to no treatment (N1) and animals which were exposed to commercially available recombinant IFN-β-1a under identical conditions (N2) were used as controls. The animals were sacrificed with CO₂ and the blood was taken as EDTA blood by cardiac puncture. After removal of cellular constituents, the serum was analyzed using a commercial huIFN-β-specific ELISA. Various amounts of commercially available recombinant IFN-β-1a (krIFN-β-1a) were measured for a calibration plot. Tables 4 and 5 below represent the resulting measurements (means from 2 measurements):

TABLE 4
Measurements for the calibration plot

| krIFN-β-1a (I.U.) | |
|---|---|
| 2.5 | 0.134 |
| 5 | 0.319 |
| 10 | 0.567 |
| 20 | 0.752 |
| 50 | 1.985 |
| 100 | 2.283 |
| 200 | 2.465 |

TABLE 5
Measurements, means and calculated amounts
after dermal administration of IFN-β-1a-TLM (TLM) and
for the controls (N1 and N2)

| | | |
|---|---|---|
| TLM | N1 | N2 |

| | 4 h | 8 h | 8 h | 4 h | 8 h |
|---|---|---|---|---|---|
| | 0.321 | 0.538 | 0.038 | 0.040 | 0.044 |
| | 0.353 | 0.647 | 0.043 | 0.044 | 0.050 |
| | 0.229 | 0.621 | 0.035 | 0.047 | 0.038 |
| | 0.355 | 0.470 | 0.040 | 0.038 | 0.047 |
| | 0.393 | 0.690 | 0.041 | 0.040 | 0.040 |
| | 0.261 | 0.767 | | 0.038 | 0.039 |
| | 0.281 | 0.735 | | | 0.048 |
| | 0.505 | 1.032 | | | 0.032 |
| | 0.401 | 0.744 | | | 0.038 |
| Mean | 0.344 | 0.694 | 0.039 | 0.041 | 0.042 |
| I.U. | 7 | 14 | 1 | 1 | 1 |

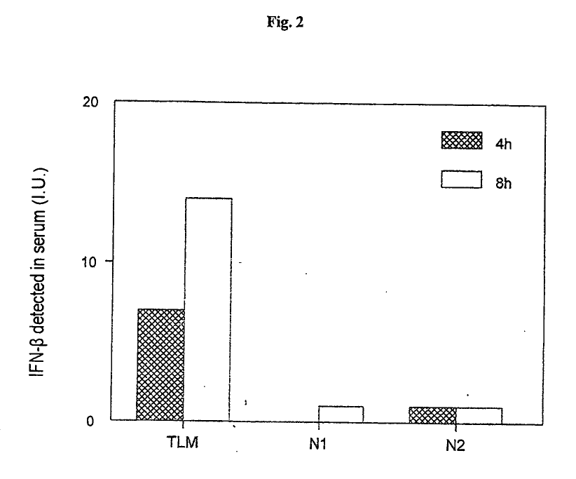

The ELISA values were averaged for each experiment and the amount detected in the serum was calculated using the calibration plot. In FIG. 2, the calculated amounts (I.U.) of the IFN-β detected in the serum are plotted in a bar diagram. FIG. 2 shows that an increased amount of IFN-β was present in the serum after dermal administration of IFN-β-1a-TLM for 4 h and 8 h, respectively, whereas no significant amount of IFN-β was detectable in the serum for the controls. The results thus show that the absorption of IFN-β through the skin is increased by the coupling of TLM to IFN-β. It surprisingly emerged that the amount of IFN-β detectable in the serum during the test period increased by a factor of 2. A depot effect such as is typical of subcutaneous administration is thus also achieved by the method of the invention without invasive administration being necessary therefor.

### Example 8

### Demonstration of the Functionality of IFN-β-1a-TLM Taken Orally

The functionality was investigated as described above using the HBV-producing cell line HepG2.2.15. The cells were spread in 24-well plates. After 24 h, the medium was changed and replaced by medium which was diluted 1:1 with the mouse sera which are identified by an asterisk in example 6, table 2 (IFN-β sera). Untreated cells (N1) and mouse serum from untreated animals (N2) served as controls. This method was repeated after 24 h and, after a further 24 h, the amount of virus in the supernatant was quantified by taqman PCR (Stoeckl et al., 2003). Table 6 indicates the resulting values (HBV genome/ml) as mean of a duplicate determination.

TABLE 6
Amounts of virus in the supernatant

| | IFN-β sera | N1 | N2 |
|---|---|---|---|
| | 4.7 × 10³ | 3.7 × 10⁶ | 2.8 × 10⁶ |
| | 9.2 × 10³ | 5.1 × 10⁶ | 4.2 × 10⁶ |
| | 2.8 × 10⁴ | 4.7 × 10⁶ | 5.1 × 10⁶ |
| | 7.5 × 10³ | 6.1 × 10⁶ | 3.4 × 10⁶ |
| | 3.8 × 10⁴ | 5.7 × 10⁶ | 2.1 × 10⁶ |
| | 1.1 × 10⁴ | | |
| | 6.3 × 10⁴ | | |
| Mean | 2.3 × 10⁴ | 5.1 × 10⁶ | 3.5 × 10⁶ |

The results show that virus propagation was reduced by 99.5% by the sera obtained from animals from example 6 treated with IFN-β-1a-TLM, whereas the sera from the untreated animals showed only a very slight antiviral effect.

### Example 9

### Demonstration of the Oral Availability of PreS1PreS2 by Feeding Tests

B6 mice (9 animals) were kept without feed for 18 h. At the start of the test, the animals received a weighed piece of toasted bread (about 3.5 to 4.5 g) which had been impregnated with 1 ml of a 200 μM PreS1PreS2 solution. The PreS1PreS2 protein comprises the HBV-TLM endogenously at its C terminus. Animals (5 animals) remained untreated as negative controls. The animals were fed for 8 h. The animals were sacrificed with CO₂ and the blood was removed as EDTA blood by cardiac puncture. After removal of cellular constituents, the serum was analyzed by Western blot analysis using a PreS1PreS2-specific serum. The Western blots showed that PreS1PreS2 protein was detectable in the serum of 9 of 9 animals, but not in the controls, under these conditions.

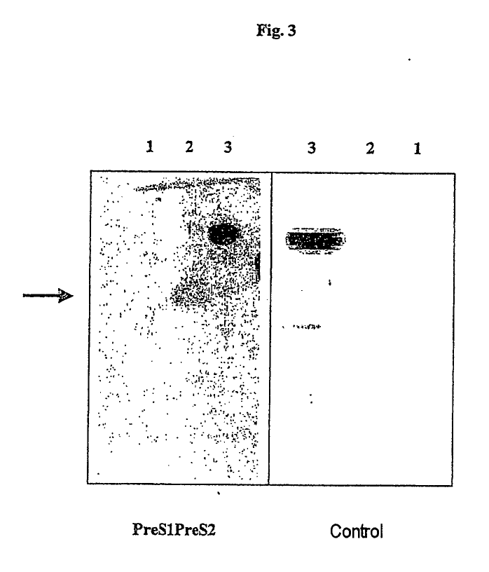

A further series of experiments investigated the extent to which oral intake of PreS1PreS2 protein can lead to the production of PreS1PreS2-specific antibodies. For this purpose, the animals were kept as described above and were fed with PreS1PreS2 protein for 14 days over a period of 4 weeks. A total of 6 weeks after the first feeding, the animals were sacrificed as described above, and the serum was obtained. Blot strips were prepared, loading one lane with cytochrome c (200 ng), one lane with PreS1PreS2 protein (20 ng) and one lane with the heavy IgG chain (marker). These strip blots were incubated with the sera obtained. The bound antibodies were detected using a peroxidase-coupled anti-mouse IgG-specific secondary antibody. In total, PreS1PreS2-specific antibodies were detectable in 9 of 9 sera. The control (cyctochrome c) showed no signal in any case, underlining the specificity of the antibodies. FIG. 3 shows two typical Western blots in this series of tests (lane 1: cytochrome c; lane 2: PreS1PreS2; lane 3: heavy IgG chain).

### Example 10

### Demonstration of the Dermal Availability of PreS1PreS2

B6 mice (9 animals) were carefully shaved in order not to injure the skin, and kept with a gauze dressing (2×6 cm, 2-layer) which was impermeable on the outside and had been impregnated in 1 ml of a 200 μM PreS1PreS2 solution for 8 h. Untreated animals (4 animals) served as controls. The animals were sacrificed with CO₂ and the blood was removed as EDTA blood by cardiac puncture. After removal of cellular constituents, the serum was analyzed by Western blot analysis using a PreS1PreS2-specific serum.

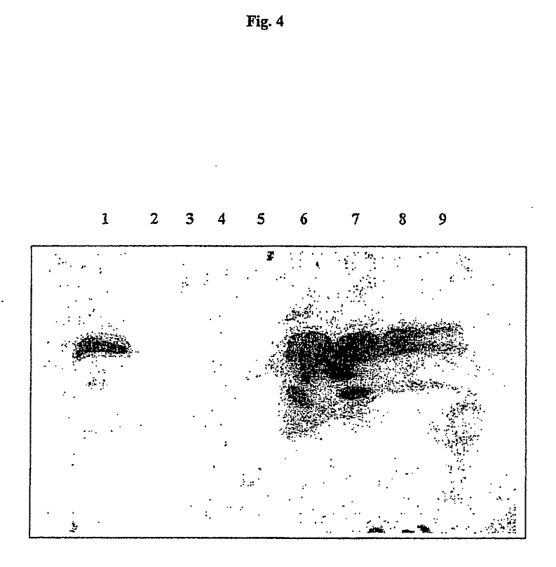

The Western blots showed that the PreS1PreS2 protein was detectable in the serum of 8 of 9 animals but not in the controls, under these conditions. FIG. 4 shows a typical example of a Western blot in this series of tests (lane 1: positive control; lanes 2 to 5: sera from untreated animals; lanes 6 to 9: sera from animals to which PreS1PreS2 was administered dermally).

1-17. (canceled)

18. A method for increasing the ability of a substance to be absorbed by skin or mucosa upon application thereto, comprising coupling (i) the substance to be absorbed to (ii) at least one agent which increases absorption of said substance through skin or mucosa.

19. The method of claim 18 wherein the step of coupling further comprises thereby increasing bioavailability of the substance to be absorbed upon application of said substance to the skin or mucosa.

20. The method of claim 18 wherein the agent which increases absorption of the substance to be absorbed comprises at least one agent selected from the group consisting of an agent which increases bioavailability of the substance and an agent which increases permeation ability of the substance.

21. The method of claim 18 wherein the substance to be absorbed is covalently coupled to the agent which increases absorption of the substance.

22. The method of claim 18 wherein the agent which increases absorption of the substance to be absorbed comprises a polypeptide or protein.

23. The method of claim 22 wherein the polypeptide or protein comprises an amino acid sequence having the formula:
X1-X2-X3-X4-X5-X6-X7-X8-X9-X10-X11-X12 (SEQ ID NO: 10)
wherein
X1, X6, X7, X9, X10 and X12 are variable amino acids,
X2 and X5 are hydrophobic amino acids, and
X3, X4, X8 and X11 are hydrophilic amino acids.

24. The method of claim 22 wherein the polypeptide or protein comprises an amino acid sequence of the formula PLSSIFSRIGDP (SEQ ID NO: 1).

25. The method of claim 18 wherein the substance to be absorbed comprises a polypeptide or a protein.

26. The method of claim 25 wherein the polypeptide or protein comprises an interferon.

27. The method of claim 26 wherein the interferon is selected from the group consisting of an interferon that is active after absorption and an interferon that is inactive after absorption.

28. The method of claim 18 wherein the substance to be absorbed comprises a virus or virus-like particle.

29. The method of claim 18 wherein the mucosa is selected from the group consisting of gastrointestinal tract mucosa, eye mucosa, nasal mucosa, tracheal mucosa, bronchial mucosa, lung mucosa, oral cavity mucosa, rectal mucosa and vaginal mucosa.

30. The method of claim 29 wherein the gastrointestinal tract mucosa is selected from the group consisting of intestinal mucosa and gastric mucosa.

31. A transdermal or transmucosal product, comprising (i) a substance to be absorbed by skin or mucosa upon application thereto; and (ii) at least one agent which increases absorption of said substance through skin or mucosa, wherein the substance of (i) is coupled to the agent of (ii).

32. The transdermal or transmucosal product of claim 31 wherein the agent which increases absorption of the substance to be absorbed comprises at least one agent selected from the group consisting of an agent which increases bioavailability of the substance and an agent which increases permeation ability of the substance.

33. The transdermal or transmucosal product of claim 31 wherein the substance to be absorbed is covalently coupled to the agent which increases absorption of the substance.

34. The transdermal or transmucosal product of claim 31 wherein the agent which increases absorption of the substance to be absorbed comprises a polypeptide or protein.

35. The transdermal or transmucosal product of claim 34 wherein the polypeptide or protein comprises an amino acid sequence having the formula:
X1-X2-X3-X4-X5-X6-X7-X8-X9-X10-X11-X12 (SEQ ID NO: 10)
wherein
X1, X6, X7, X9, X10 and X12 are variable amino acids,
X2 and X5 are hydrophobic amino acids, and
X3, X4, X8 and X11 are hydrophilic amino acids.

36. The transdermal or transmucosal product of claim 34 wherein the polypeptide or protein comprises an amino acid sequence of the formula PLSSIFSRIGDP. (SEQ ID NO: 1)

37. The transdermal or transmucosal product of claim 31 wherein the substance to be absorbed comprises a polypeptide or a protein.

38. The transdermal or transmucosal product of claim 37 wherein the polypeptide or protein comprises an interferon.

39. The transdermal or transmucosal product of claim 38 wherein the interferon is selected from the group consisting of an interferon that is active after absorption and an interferon that is inactive after absorption.

40. The transdermal or transmucosal product of claim 31 wherein the substance to be absorbed comprises a virus or virus-like particle.

41. A pharmaceutical composition comprising the transdermal or transmucosal product of claim 31 and a pharmaceutically acceptable carrier.

42. A pharmaceutical composition comprising the transdermal or transmucosal product of claim 31 and a pharmaceutically acceptable carrier, wherein the pharmaceutical composition is suitable for topical or oral administration.

43. A method of treating a patient with a substance to be absorbed by skin or mucosa upon application thereto, comprising administering to the skin or mucosa of said patient a composition comprising the transdermal or transmucosal product according to any one of claims 31-40.

44. The method of claim 43 wherein the mucosa is selected from the group consisting of gastrointestinal tract mucosa, eye mucosa, nasal mucosa, tracheal mucosa, bronchial mucosa, lung mucosa, oral cavity mucosa, rectal mucosa and vaginal mucosa.

45. The method of claim 44 wherein the gastrointestinal tract mucosa is selected from the group consisting of intestinal mucosa and gastric mucosa.
